(19) United States
(12) Patent Application Publication
(10) Pub. No.: US 2025/0128055 A1
(43) Pub. Date: Apr. 24, 2025

(54) TREATMENT OF PSYCHIATRIC DISORDERS WITH DEEP BRAIN STIMULATION

(71) Applicant: Albert Ludwigs Universität Freiburg, Freiburg im Breisgau (DE)

(72) Inventors: Volker Coenen, Freiburg im Breisgau (DE); Thomas Schläpfer, Freiburg im Breisgau (DE)

(73) Assignee: Albert Ludwigs Universität Freiburg, Freiburg im Breisgau (DE)

(21) Appl. No.: 18/691,645

(22) PCT Filed: Oct. 7, 2022
(86) PCT No.: PCT/IB2022/059612

§ 371 Date: Mar. 13, 2024

Related U.S. Application Data
(60) Provisional application No. 63/253,740, filed on Oct. 8, 2021.

(51) Int. Cl. A61N 1/05; A61N 1/36

(52) U.S. Cl. CPC A61N 1/0534; A61N 1/36078; A61N 1/36096; A61N 1/36117

(57) ABSTRACT

Provided is a method of treating a psychiatric disorder and/or blood pressure disorder in a subject in need thereof. The method includes implanting one or more leads into the subject, wherein each of the one or more leads comprises a plurality of electrodes; generating an electrical signal; generating an electrical field that is substantially directional and substantially focused; and delivering the electrical field to a superolateral branch in a medial forebrain bundle of the subject via at least one of the plurality of electrodes. Provided are also an implantable pulse generator configured to deliver the corresponding electrical field via respective leads and electrodes.

### CROSS-REFERENCE TO RELATED PATENT APPLICATIONS

This application claims the benefit of priority to U.S. Provisional Patent Application No. 63/253,740, filed Oct. 8, 2021, which is hereby incorporated by reference in its entirety for any and all purposes.

### BACKGROUND OF THE DISCLOSURE

Approximately 20% of the U.S. population is living with a psychiatric disorder. The most common psychiatric disorders are mood disorders and anxiety disorders such as depression, bipolar disorder, panic disorder, generalized anxiety disorder, phobia, separation anxiety disorder, and obsessive-compulsive disorder (“OCD”). Anxiety disorders likely stem for evolutionarily useful reactions and behaviors, but in excess, these reactions and behaviors can lead to highly impairing psychiatric disorders. For example, OCD is characterized by intruding, unpleasant thoughts (obsessions) and/or repetitive, rigid behaviors (compulsions) (DSM-5). If these thoughts and/or behaviors reach pathological value, OCD means enormous suffering such as strong impairments in social functioning, often accompanied by at least one comorbid psychiatric disorder and may even culminate in suicidality.

### SUMMARY OF THE DISCLOSURE

The present disclosure provides a method of treating a psychiatric disorder and/or blood pressure disorder in a subject in need thereof. The method includes implanting one or more leads into the subject, wherein each of the one or more leads comprises a plurality of electrodes; generating an electrical signal; generating an electrical field; and delivering the electrical field to a superolateral branch in a medial forebrain bundle (“slMFB”), VMT, or VTA of the subject via at least one of the plurality of electrodes. Preferably, at least one or more of the electrical fields is substantially directional (e.g., not omnidirectional) and substantially focused. At least 50% of the electrical fields can be substantially directional (e.g., not omnidirectional) and substantially focused. All of the electrical fields can be substantially directional (e.g., not omnidirectional) and substantially focused.

The directionality of the electrical field can at least 50°. The focus of the electrical field can have a focal radius of ≤4 mm and/or the electrical field may exhibit a volume of tissue activated of 1 mm³ to 50 mm³. The psychiatric disorder can include depression, sleep disorder, anxiety disorder, anorexia nervosa, post-traumatic stress disorder, or a combination of any two or more thereof.

The one or more leads can be surgically implanted into the medial forebrain bundle (MFB). The method can include delivering the electrical field to the MFB via at least one of the plurality of electrodes. The method can include delivering the electrical field to the superolateral medial forebrain bundle (slMFB), VMT, or VTA (e.g., unique landing zone) via at least one of the plurality of electrodes. The neural tissue near the distal end of the one or more leads can be stimulated with the electrical field generated by the electrical signal (e.g., neural target tissue). The electrical signal may be transmitted from an implantable pulse generator (e.g., implantable stimulator). The method may further include recording neurological activity from the MFB, and selecting a portion of the plurality of electrodes to deliver the electrical signal based on the recorded neurological activity. The electric signal (e.g., electrical stimulation) may be carried out continuously or intermittently. The substantially directional and substantially focused electrical field may reduce side effects caused by the electrical stimulation.

The one or more leads may further include a MEMS film that includes the plurality of electrodes. Additionally, the MEMS film may include a plurality of periphery traces at least partially encircling each of the plurality of electrodes and/or at least two connection points coupling each of the plurality of periphery traces with a respective one of the plurality of electrodes.

The foregoing summary is illustrative only and is not intended to be in any way limiting. In addition to the illustrative aspects, embodiments and features described above, further aspects, embodiments and features will become apparent by reference to the following drawings and the detailed description.

### BRIEF DESCRIPTION OF THE DRAWINGS

The figures, described herein, are for illustration purposes. In some instances various aspects of the described implementations may be shown exaggerated or enlarged to facilitate an understanding of the described implementations. In the drawings, like reference characters generally refer to like features, functionally similar and/or structurally similar elements throughout the various drawings. The drawings are not necessarily to scale, emphasis instead being placed upon illustrating the principles of the teachings. The drawings are not intended to limit the scope of the present teachings. The systems and methods may be better understood from the following illustrative description with reference to the following drawings in which:

FIG. 1 illustrates an example system for treating psychiatric and/or blood pressure disorders.

FIG. 2 illustrates the example lead for use in the system illustrated in FIG. 1.

FIGS. 3A-3B illustrate the distal end of the lead for use in the system illustrated in FIG. 1.

FIG. 4 illustrates a block diagram of the components of an implantable pulse generator (e.g., implantable stimulator) for use in the system illustrated in FIG. 1.

FIG. 5 illustrates a flow chart of a method for tuning the electrical stimulation delivered to a patient using the system illustrated in FIG. 1.

FIG. 6 illustrates an artistic representation of the slMFB stimulated region and the surrounding structures.

FIGS. 7A-7D illustrate the neural reward network 750 driven in part by the slMFB 755 and the neural affect network 770 driven by the ATR 765, which converge onto the prefrontal cortex 705 to form the cortico-striato-thalamo-cortical (CSTC) loop 785. FIGS. 7A-B show a quasi-anatomical schematic of a brain 140 and FIGS. 7C-D show schematics illustrating the CSTC loop 785 before (FIG. 7A or C) and after (FIGS. 7B and D) the DBS treatment of the present disclosure.

FIGS. 8A-8C show coronal and axial view of template contacts 805 illustrating implanted electrode 160 positions in the unique landing zone 1060 identified herein (e.g., the mesencephalic ventral tegmentum (VMT), VTA 725 or slMFB 755) for treating OCD in patients. FIGS. 8A and 8B show magnetic resonance imaging (MRI) images coronal (FIG. 8A) and axial (FIG. 8B) view of template contacts 805 with electrode 160 contacts implanted in the patients. FIG. 8C show a three-dimensional reconstruction of template contacts 805 with implanted active electrodes 160 with effectively stimulated contacts three months after stimulation onset and non-responder electrodes 810.

FIGS. 9A-9C are graphs illustrating the relative changes of the symptom severity over time for each patient. FIGS. 9A and 9B are graphs illustrating the relative changes of OCD symptom severity from a clinician rating (FIG. 9A) and from a patient rating (FIG. 9B). FIG. 9C is a graph illustrating the relative changes of depressive symptoms from a clinician rating.

FIGS. 10A-10C show schematic 1000 illustrating an overview of the medial forebrain bundle 1000 (MFB (755); Maintenance system) identified herein as being essential for treating OCD with Deep brain stimulation with minimal to no side effects and part of the motor control systems. FIG. 10A shows a schematic of a brain 140 with the Maintenance circuitry 1020 and 1015. FIG. 10B shows a schematic illustrating an enlarged midbrain 1030 topographic overview of the unique landing zone 1060 for DBS lead 130 or electrode 160 placement identified herein. FIG. 10C shows a schematic illustrating the midbrain 1030 topographic overview illustrating the slMFB fibers (755) and electrode positions (1070) at the unique landing zone (1060) for OCD treatment described herein.

FIG. 11 shows a postoperative computed tomography three-dimensional reconstruction of Deep brain stimulation (DBS) 1100 illustrating the position of two DBS leads 130 with respect to the STN (735), which is outside the unique landing zone for OCD treatment described herein.

FIGS. 12A-12C show postoperative computed tomography three-dimensional (3-D) reconstruction images 1200 of a simulation of Deep brain stimulation (DBS) settings 1215 in a patient suffering from dyskinesia before (FIGS. 12A-B) and after (FIG. 12C) electrode reprograming 1210 to fully avoid the amSTN 735 and focus on the VMT, VTA 725 and slMFB 755 (landing zone 1060).

FIG. 13 shows a schematic 1300 illustrating that DBS of the slMFB 0755 specifically targets and reduces the obsession component 1305 (e.g., emotional consequences) of OCD without affecting its compulsion component 1310 (e.g., motor consequences of the behavior).

FIG. 14 shows a table illustrating pre-operative assessments of various subjects.

FIGS. 15A-B show a table illustrating pre-operative and post-operative clinical measurements of various subjects.

### DETAILED DESCRIPTION

There is a high prevalence of psychiatric disorders in the U.S. as well as around the world with the most common psychiatric disorders being mood disorders and anxiety disorders. The first line treatment for several of these disorders is selective serotonin inhibitors (SSRIs) and psychotherapy treatment (e.g., cognitive behavioral therapy). However, some subjects do not respond sufficiently to these first line treatments. For these patients, other treatments are needed.

An accepted treatment strategy for common psychiatric disorders (e.g., refractory depression (TRD) or obsessive-compulsive disorders (OCD)) is deep brain stimulation (DBS) of a subsection of the medial forebrain bundle (MFB), known as the superolateral medial forebrain bundle (slMFB). The MFB is an anato-physiologically region of the brain that regulates emotion (reward system), and controls emotion associated bodily reactions, motor display of positive affect and motor learning. In particular, the MFB is associated with the seeking system, which promotes euphoric drive, reward anticipation and reward. This seeking system is mediated in part by the activation of the mesocorticolimbic dopaminergic system.

Therapy refractory depression (TRD) and obsessive-compulsive disorder (OCD) may be characterized by deficiencies in showing flexible behavior. Indeed, OCD may be associated with a deficiency in the automated classification of emotion related information (e.g., affect valence). As such, psychiatric diseases like OCD and TRD may be considered as network dysfunctions of the MFB. Inventors of the present disclosure unexpectedly found that, in addition to regulating the reward system, the MFB may also serve an overarching role in the maintenance system. The maintenance system governs the valence of basal emotional states (e.g., aversive vs. appetitive) and serves to drive emotional arousal, basic and higher affect valence, bodily reactions, motor programs, vigor and flexible behavior. This regulatory role may be the basis of the high efficiency of deep brain stimulation (DBS) of the superolateral medial forebrain bundle (slMFB) as described herein. The overarching role of the slMFB in the maintenance system can be the basis for the antidepressant and anti-OCD efficacy of deep brain stimulation of the unique landing zone in the VMT/VTA described herein.

Indeed, the present inventors have identified an ideal neural area (e.g., unique landing zone) for implanting leads and electrodes for DBS stimulation within the VMT/VTA (e.g., slMFB) that when stimulated with deep brain stimulation significantly reduced OCD symptoms when compared to stimulation in other areas of the brain. In addition to the suppression of OCD symptoms, stimulation of this ideal area within the VMT/VTA also resulted in the suppression of depressive symptoms. The suppression of the symptoms can occur within about 1 hour, about 2 hours, about 3 hours, about 4 hours, about 5 hours, about 6 hours, about 7 hours, about 8 hours, about 9 hours, about 10 hours, or at least 24 hours. Alternatively, the suppression of the symptoms can occur within about 1 day, about 2 days, about 3 days, about 4 days, about 5 days, about 6 days, about 7 days, about 8 days, about 10 days, about 15 days, about 20 days, about 25 days, or about 30 days. The identification of this ideal neural area (e.g., unique landing zone) for implanting leads and electrodes for DBS stimulation within the VMT/VTA (e.g., slMFB) supports the idea of a shared disease-network and indicated that deep-brain stimulation of the ideal neural area or unique landing zone for electrode placement may result in the normalization of the reward network induced by the modulation of the slMFB-activity.

The ideal neural area or unique landing zone for DBS OCD treatment described herein (e.g., target region) is located in the corridor between red nucleus, substantia nigra/subthalamic nucleus and the mammillothalamic tract. Stimulation related side effects such as stimulation-induced dyskinesia were completely inhibited by steering the electrodes away from the anteromedial subthalamic nucleus (amSTN) medial into the direction of the mesencephalic ventral tegmentum (VMT). The MTV contains the ventral tegmental area (VTA) and slMFB. The present disclosure shows for the first time that an anti-OCD network which can be targeted by DBS with no side effects (e.g., dyskinesia) may be located outside and medial to the subthalamic nucleus (STN). The anti-OCD network which can be targeted by DBS with little or no side effects (e.g., dyskinesia) may be located in the VMT, preferably in the VTA, and more preferably in the slMFB.

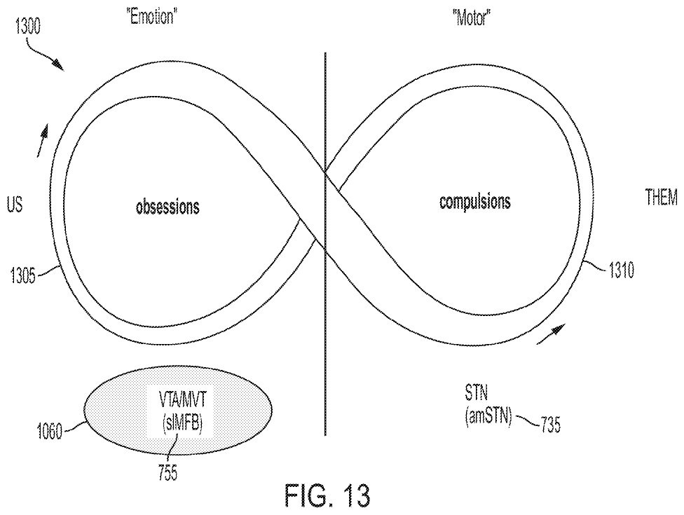

In addition, the present disclosure shows that a distinct neural circuit modulates the emotional component of OCD (e.g., the VMT/slMFB) and a different neural circuit modulates the compulsion component of OCD (e.g., amSTN). These distinct pathways are illustrated in FIG. 13, which shows a model whereby the PRF, VMT, VTA, and/or slMFB specifically may modulate the obsession component (emotional consequences) of OCD without affecting its compulsion component (motor consequences of the behavior); whereas the amSNT may control the motor consequences of these emotions. The system and the method of treatment disclosed herein can suppress intrusive (e.g., patients undergoing a DBS treatment for OCD disclosed herein no longer overreact when having intrusive thoughts). Negative symptoms of OCD can also be suppressed, as well as anxiety.

Accordingly, one aspect of the present disclosure provides a method of treating one or more psychiatric disorders in a subject in need thereof comprising the DBS stimulation of the SIMFB, VTA, VMT, or pre-rurbral fields (PRF) to specifically target and reduce the obsession component (emotional consequences) of OCD without affecting its compulsion component (motor consequences of the behavior). DBS cannot stimulate the amSTN.

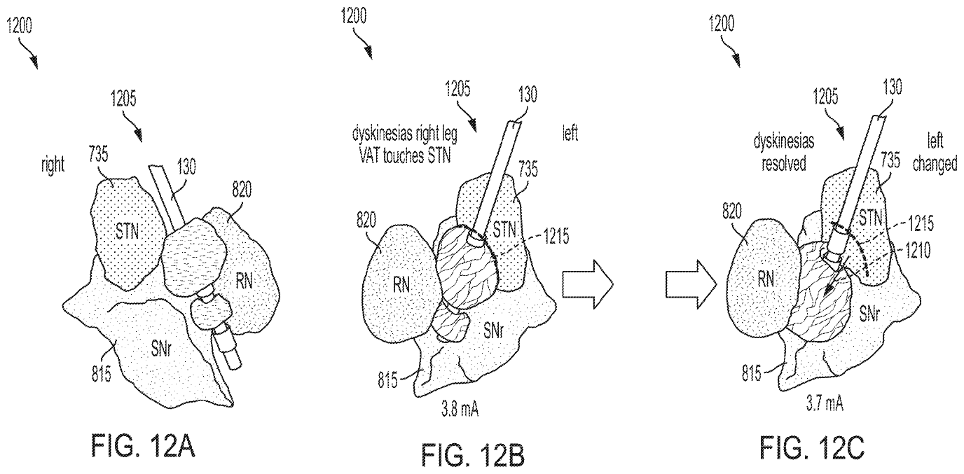

Another aspect of the present disclosure provides a method of treating a psychiatric disorder in a subject in need thereof by electrical stimulation of the slMFB, VMT or VTA. The method of treatment disclosed herein is based on the placement of DBS electrodes on a newly identified, very specific, and unique neural location in the VMT/VTA network (e.g., ideal neural area, landing zone, or unique landing zone). The present specification describes this landing zone and shows for the first time the efficacy of this landing zone based on clinical information (e.g., Example 3). The ideal neural area or unique landing zone can be or can be within the mesencephalic ventral tegmentum (VMT), the slMFB, or pre-rurbral fields (PRF). Alternatively, the ideal neural area or unique landing zone can be within the mesencephalic ventral tegmentum (VMT), the slMFB, or pre-rurbral fields (PRF). The unique landing zone or ideal neural area identified herein can be anatomically unambiguous from the STN. The unique landing zone or ideal neural area cannot be the STN or the anteromedial STN (amSTN). The unique landing zone or ideal neural area can regulate the emotion component (e.g., obsession) of OCD. Indeed, as shown in FIGS. 12A-C, turning the DBS electrodes and stimulation away from the amSTN toward the VMT maintains anti-OCD effects when compared to the stimulation of the amSTN and/or other region of the brain. IDBS stimulation of the VMT, VTA and/or slMFB can treat one or more psychiatric disorders described herein.

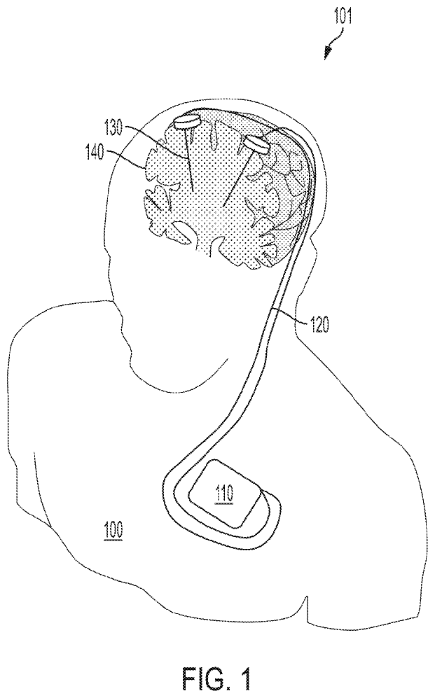

The present disclosure provides a method of treating a psychiatric disorder and/or blood pressure disorder in a subject in need thereof by electrical stimulation of the brain. One aspect the present disclosure provides a method of treating a psychiatric disorder using deep brain stimulation of the superolateral branch of a medial forebrain bundle (slMFB) VTA, or VMT. The method includes implanting one or more leads into the subject, wherein each of the one or more leads comprises a plurality of electrodes; generating an electrical signal; generating an electrical field; and delivering the electrical field to a superolateral branch of a medial forebrain bundle (slMFB), VTA, or VMT of the subject via at least one of the plurality of electrodes. Preferably, the electrical field is substantially directional and substantially focused. FIG. 1 illustrates an example implantable electrical system 101 for treating a psychiatric disorder and/or blood pressure disorder comprising an implantable pulse generator (IPG) and one or more stimulation leads, as described herein. The system 101 includes an implantable pulse generator (IPG) 110 (e.g., implantable stimulator) implanted in the chest of a patient 100 (e.g., subject). The IPG 110 can be implanted into the patient's clavicle area or in other areas. An extension cable 120 couples the IPG 110 to one or more leads 130 comprising a plurality of electrodes. The one or more leads 130 are each coupled to the IPG 110 by an extension cable 120 (or a plurality of extensions 120) can be implanted in the patient 100. As illustrated, the one or more leads 130 are implanted into the brain 140 of the patient 100.

The system 101 can include the IPG 110 (e.g., control module). The IPG 110 is configured to generate electrical signals transferred through the extension cable 120 to the one or more leads 130 comprising the plurality of electrodes to generate an electrical field that stimulates the neural target tissue. In some implementations, the IPG 110 is also configured to record electrical activity generated by the neural target tissue and detected by the one or more leads 130. The IPG 110 includes pulse generation circuitry that delivers electrical stimulation energy in the form of, for example, a pulsed electrical waveform (e.g., a temporal series of electrical pulses) to an electrode array in accordance with a set of preselected stimulation parameters.

The IPG 110 can have multiple stimulation channels which may be independently programmable to control the magnitude of the current stimulus from each channel. The IPG 110 can have any suitable number of stimulation channels including, but not limited to, 4, 6, 8, 12, 16, 32, or more stimulation channels.

The IPG 110 can be configured to supply a range of electrical signals to neural target tissue by adapting a pulse frequency, a pulse width, a pulse amplitude, or any combination thereof. The IPG 110 can generate pulse frequency ranges between about 2 Hz and about 1 kHz, between about 2 Hz and about 500 Hz, between about 2 Hz and about 250 Hz, between about 25 Hz and about 225 Hz, between about 50 Hz and about 200 Hz, between about 75 Hz and about 200 Hz, between about 100 Hz and about 160 Hz, between about 110 Hz and about 150 Hz, or between about 120 Hz and about 140 Hz. Pulse widths ranges can be between about 1 μs and about 1000 μs, between about 1 μs and about 500 μs, between about 10 μs and about 500 μs, between about 20 μs and about 150 μs, between about 80 μs and about 120 μs, between about 20 μs and about 100 μs, or between about 20 μs and about 50 μs.

The pulse amplitudes can be at least 0.01 mA. The IPG 110 can be current driven, and the pulse amplitudes may be at least 0.05 mA. The pulse amplitudes can range from between about 0.05 mA to about 15 mA, between about 0.05 mA and about 12 mA, between about 0.05 mA to about 6 mA, between about 0.1 mA and about 3 mA, between about 1 mA to about 3 mA, or between about 1.5 mA and about 5.5 mA. The IPG 110 can be voltage driven, and the pulse amplitude may be between about 0.1 V and about 10 V or between about 2 V and about 4 V.

The stimulation can be continuous, for example lasting days, weeks, months, or years. Over the course of the continuous stimulation, the stimulation can be delivered intermittently. For example, the stimulation can be provided for 10 minutes every hour over the course of 1 month. These ranges are examples and other ranges are possible. The stimulation parameters can be subject (e.g., patient) or disease specific and can vary over the course of the patient's treatment. For example, the stimulation parameters can be increased over time if the patient's body begins to encapsulate the electrodes of the electrode lead 130. Different stimulation parameters may induce different neurological responses in the patient, including improved or decreased beneficial effects and decreased side effects. In some implementations, the IPG 110 is configured to excite neural activity (also referred to increasing neural activity) at the brain target or inhibit neural activity (also referred to as decreasing neural activity) at the brain target.

The IPG 110 can be configured to capture and record signals from the brain or other target tissue. The captured signals can be analyzed to determine if the signals are indicative of a disease state. For example, in some neurological disease states, it may be possible to determine a brain volume directly affected by the disease state by its lack of neurophysiological activity, or inversely by its overactive neurophysiological activity. By performing recordings from the distal end 150 of the electrode lead 130, neurophysiological marker signals can be recorded and analyzed by a machine learning algorithm to determine if the disease state is present. Thresholds can be set to indicate whether the neurophysiological activity is in an inactive state or an active state. The recorded signals also can be presented to the physician via a telemetric connection with the IPG 110. The physician can make a decision as to which electrodes of the electrode lead 130 is best placed to use for therapeutic stimulation. In some implementations, the IPG 110 includes a signal processing algorithm that independently determines which electrodes of the electrode lead 130 to use to deliver the electrical stimulation to without physician intervention. This can be referred to herein as a closed-loop stimulation.

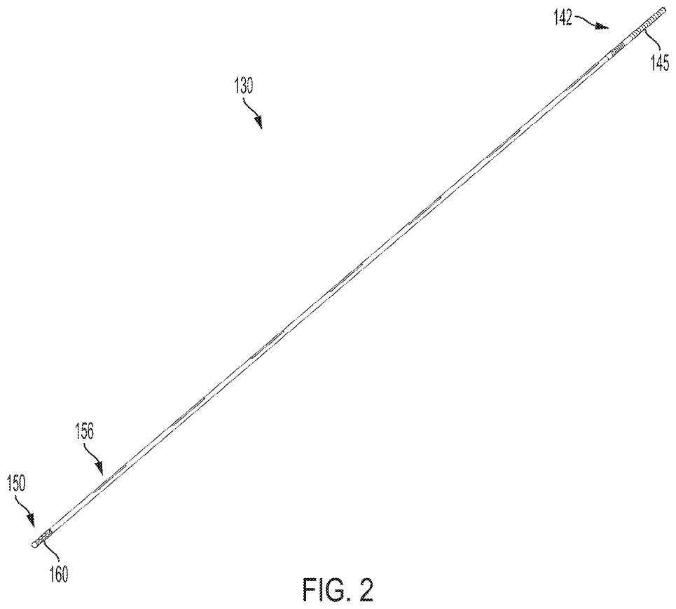

FIG. 2 illustrates an example stimulation lead 130. The stimulation lead 130 includes a body. The body may also be referred to as a tube body, tube, or catheter. The body includes several orientation markers 156. At a distal end 150, the stimulation lead 130 includes a MEMS film comprising a plurality of electrodes 160. At a proximal end 142, the stimulation lead 130 includes a plurality of contacts 145. At the proximal end 142 of the stimulation lead 130, the stimulation lead 130 includes one or more contacts 145. The contacts 145 can be used to establish an electrical connection between the electrodes 160 of the MEMS film and the IPG 110. For example, each of the contacts 145 can be coupled with one or more electrodes 160 of the MEMS film via lead wires that run the length of the stimulation lead 130. A stimulator may be coupled with the contacts 145 through a plurality of cables 120 to stimulate tissue or record physiological signals. The implantable electrical system can comprise two, three, four, or more leads 130 coupled to the IPG 110. The distal end 150 of the stimulation lead 130 can include a MEMS film that includes a plurality of electrodes 160.

The one or more leads may have a MEMS film further comprising a plurality of periphery traces at least partially encircling each of the plurality of electrodes and at least two connection points coupling each of the plurality of periphery traces with a respective one of the plurality of electrodes. The distal end cannot include a MEMS film but is implemented using common manufacturing methods. The electric fields generated by the plurality of electrodes may be omni-directional and/or direction, preferably at least some of the electric fields can be directional.

The distal end 150 of the lead 130 can have a diameter between about 1 mm and about 1.5 mm (e.g., +/−10%). In some implementations, the electrode lead 130 can have the same diameter along its length. A substantial portion (e.g., between about 60% and about 95%) of the lead 130 can be hollow, enabling a rigid stylet to provide support to the lead 130 during the implantation procedures. The stylet can be removed during the surgery once the lead 130 is positioned at its final target. The lead 130 can be implanted in its target position through a surgically prepared hole in the skull. Each hemisphere of the brain can receive at least one lead 130. Each of the leads 130 is coupled to the IPG 110 via an extension cable 120 (or one or more extension cables 120).

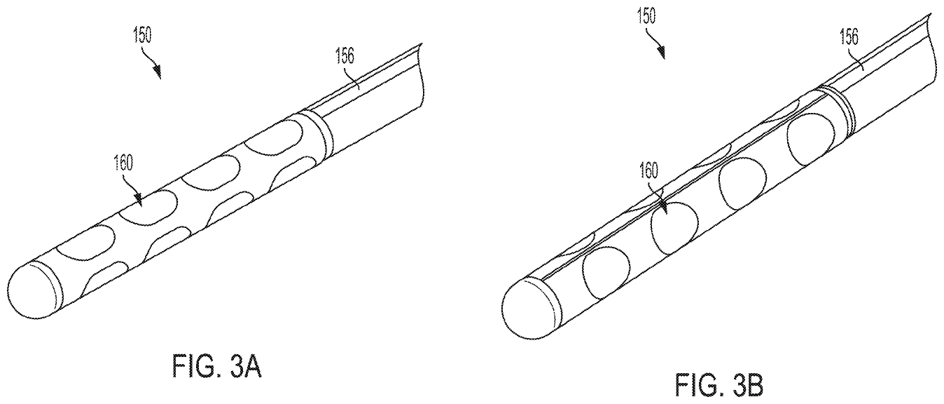

FIGS. 3A-3B illustrate an example of the distal end 150 of the lead 130, in greater detail. The distal end 150 of the lead 130 includes a plurality of segmented electrodes 160. The electrodes 160 can be formed using any conductive, biocompatible material. Examples of suitable materials include metals, alloys, conductive polymers, conductive carbon, and the like, as well as combinations thereof. For example, one or more of the electrodes 160 are formed from one or more of: platinum, platinum iridium, palladium, palladium rhodium, or titanium. The electrodes 160 of the one or more lead 130 bodies are typically disposed in, or separated by, a non-conductive, biocompatible material such as, for example, silicone, polyurethane, polyetheretherketone (“PEEK”), epoxy, and the like or combinations thereof. Electrically conductive wires, cables, or the like (not shown) extend from the terminals to the electrodes 160. Typically, one or more electrodes 160 are electrically coupled to each terminal. Each terminal can only be connected to one electrode 160.

The distal end 150 may include between 1 and 8 columns of electrodes (e.g., segmented electrodes), with each column including between 1 and 10 electrodes 160. For example, the distal end 150 may include 2-50, 2-40, 2-30, 2-20, 2-12, or 2-10 electrodes (e.g., segmented electrodes). The distal end 150 can include 2, 3, 4, 5, 6, 7, 8, 9, 10, 11, 12, 13, 14, 15, 16, 17, 18, 19, or 20 electrodes (e.g., segmented electrodes). The distal end 150 can include 4, 5, 6, 7, 8, 9, 10, 11, or 12 electrodes. The distal end 150 can include 4, 6, 8, 9, 10, or 12 electrodes.

Each of the electrodes 160 has a length along the distal end 150 of between about 0.25 mm and about 2 mm. The electrodes 160 may have any suitable longitudinal length including, but not limited to, 2, 3, 4, 4.5, 5, or 6 mm. The longitudinal spacing between adjacent electrodes 160 may be any suitable amount including, but not limited to, 1, 2, or 3 mm. As used herein, the term spacing refers to the distance between the nearest edges of two adjacent electrodes. The spacing can be uniform between longitudinally adjacent electrodes along the length of the lead 130. The spacing between longitudinally adjacent electrodes may be different or non-uniform along the length of the lead.

The distal end 150 can include eight electrodes 160 in total, which may include one or more directional electrodes, one or more omnidirectional electrodes, or a combination thereof. As illustrated in FIGS. 3A-3B, the electrodes 160 may be configured in three columns around the circumference of the distal end 150. Each column of electrodes 160 may include four electrodes 160. To provide a total of twelve electrodes 160 on the distal end 150. The electrodes 160 are substantially rounded illustrating that the electrodes are configured as directional electrodes. The electrode can have round corners. The electrode can be oval, elliptical, or circular shaped. As illustrated in FIG. 3A, the distal end 150 can include oval or elliptical shaped electrodes 160. FIG. 3B illustrates each of the electrodes 160 configured in a circular shape. Each of the electrodes 160 can roughly cover an arc angle around the circumference of the distal end 150 of about 90 degrees (e.g., +/−10 degrees).

In some implementations, each of the electrodes 160 have a length along the distal end 150 of between about 0.25 mm and about 2 mm. Each of the electrodes 160 can be individually addressed by the IPG 110 to enable directional stimulation and/or recording. Directional electrodes can enable the targeted electrical stimulation to a predetermined volume avoiding other volumes of the brain thereby reduce side effects.

In other implementations, the electrodes 160 can be square in shape or have any other polygonal shape including those disclosed in WO 2010/014686, WO 2010/055421, WO 2011/067297, WO 2011/121089, WO 2015/173787, WO 2016/030822, WO 2017/134587, WO 2019/166994, and WO 2019/207449, each of which is herein incorporated by reference. The one or more electrodes 160 may be, tip electrodes, ring electrodes, segmented electrodes, or a combination thereof.

The one or more electrodes 160 can be segmented electrodes that extend only partially around the perimeter (for example, the circumference) of the lead 130. These segmented electrodes can be provided in sets of electrodes, with each set having electrodes circumferentially distributed about the lead at a particular longitudinal position. Each of the plurality of electrodes is segmented and does not have sharp corners.

The segmented electrodes 160 can be in sets of three (one of which is not visible in FIGS. 3A-B). The three segmented electrodes of a particular set may be electrically isolated from one another and circumferentially offset along the lead 130. Any suitable number of segmented electrodes 160 can be formed into a set. For example, a set of segmented electrodes 160 may include 2, 3, 4, 6, 8, 10, or more segmented electrodes.

Segmented electrodes are particularly important for the method disclosed herein because segmented electrodes can be used to direct stimulus current to one side, or even a portion of one side, of the lead. Segmented electrodes provide precise three-dimensional targeting and delivery of the current stimulus to neural target tissue (e.g., slMFB, VMT, or VTA), while potentially avoiding stimulation of other tissue (e.g., STN or amSTN). When segmented electrodes are used in conjunction with an implantable pulse generator that delivers current stimulus, current steering can be achieved to more precisely deliver the stimulus to a position around an axis of the lead 130 (e.g., radial positioning around the axis of the lead).

Segmented electrodes may provide for superior current steering than ring electrodes in the method disclosed herein because the neural structures (e.g., slMFB; VMT, or VTA) may not be symmetrical to the axis of the distal electrode array and/or the may be too close to motor control systems (e.g., STN or amSTN). In some cases, the targeted structure (e.g., SIMFB, VMT, or VTA) may be located on one side of a plane running through the axis of the lead 130. For these reasons, current steering can be performed not only along the length of the lead but also around the perimeter of the lead, when one or more segmented electrodes 160 arrays are used.

Any number of segmented electrodes 160 may be disposed on the lead 130 body. The lead 130 may include 1, 2, 3, 4, 5, 6, 7, 8, 9, 10, 11, 12, 13, 14, 15, 16, 17, 18, 20, 25, 28, 32, or more segmented electrodes 160. Any suitable number of segmented electrodes 160 may be disposed along the length of the lead 130 body.

A segmented electrode 160 may extend about 2%, about 3%, about 5%, about 10%, about 15%, about 17%, about 18%, about 20%, about 25%, about 30%, about 35%, about 40%, about 45%, about 50%, about 55%, about 60%, about 65%, about 70%, or about 75% around the circumference of the lead.

The segmented electrodes may be grouped into sets of segmented electrodes. For example, each set may be disposed around the circumference of the lead 130 at a particular longitudinal portion of the lead 130. The segmented electrodes may also be uniform, or vary, in size and shape. The segmented electrodes can all be of the same size, shape, diameter, width, or area or any combination thereof. A lead (e.g., a lead 130) comprising electrodes 160 (e.g., segmented electrodes) may be a directional lead that can provide stimulation in a particular direction using the electrodes (e.g., segmented electrodes).

The distal end 150 can include a combination of omnidirectional electrodes and directional electrodes. The distal end 150 also includes orientation markers 156. A surgeon may orient the orientation marker 156 normal to a known plane, such as the sagittal plane, or along a known plane to enable the surgeon to know in which direction each of the electrodes 160 is facing.

In some implementations, the stimulation (both the characteristics of the stimulation signal and the election of which electrodes 160 to use in the stimulation processed) are tuned based on bio-feedback. For example, a patient may experience relief from their disorder symptoms, but experience a side effect. Therefore, the physician can choose to decrease the pulse amplitude on an active electrode until the side effects diminish, but the beneficial effect remains. This trial-and-error procedure can provide better electrode selection, pulse frequencies, pulse widths, and pulse amplitudes. A various time intervals (e.g., days, weeks, or years), the trial-and-error procedure enables updating of the stimulation parameters as needed to treat the disorder.

The electrical signal can generate an electrical field delivered to the target brain tissue (e.g., VMT, VTA, or slMFB of the subject). The directionality of the electrical field can be at least 50°. The directionality of the electrical field can be at least 40°. The directionality of the electrical field is 20° to 50° or 25° to 35°. The focus of the electrical field can have a focal radius of ≤4 mm. The focus of the electrical field can have a focal radius of 0.5 mm to 3.5 mm or 1.5 mm to 2.5 mm. The electrical field can exhibit a volume of tissue activated of 1 mm³ to 50 mm³. The electrical field can exhibit a volume of tissue activated of 1 mm³ to 50 mm³, 1 mm³ to 40 mm³, 1 mm³ to 35 mm³, 1 mm³ to 25 mm³, 2 mm³ to 20 mm³, or 4 mm³ to 10 mm³. The pulse frequency, pulse width, pulse amplitude, or any combination thereof may be adapted to provide the appropriate focal radius and/or volume of tissue activated.

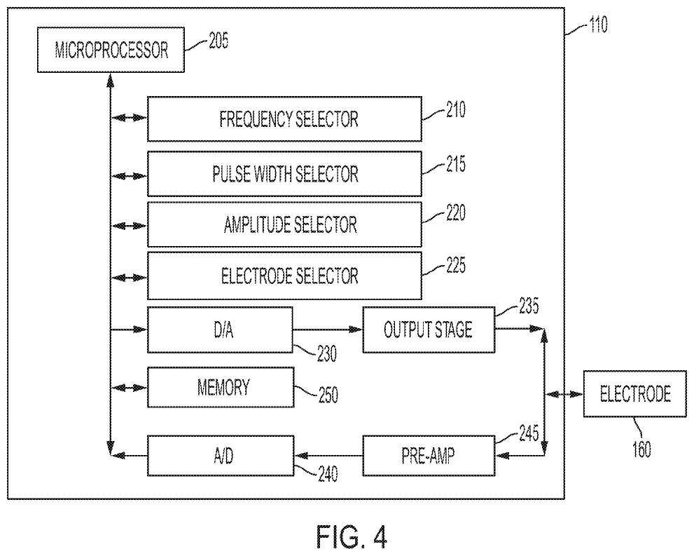

FIG. 4 illustrates a block diagram of the components of the IPG 110. The IPG 110 can include a microprocessor 205 that can coordinate and control the function of the IPG 110. The microprocessor 205 can execute any script, file, program, application, set of instructions, or computer-executable code that is stored in the memory 250, which can cause the microprocessor 205 to perform the functions of the components of the IPG 110. The IPG 110 can include a frequency selector 210. The frequency selector 210 can select and adjust the frequency of the electrical stimulation used to stimulate the target tissue. The IPG 110 also includes a pulse width selector 215 that can select and adjust the pulse width of the electrical stimulation. For example, the pulse width selector 215 can select a pulse width of the electrical stimulation between about 10 μS and about 500 μS. The IPG 110 can also include an amplitude selector 220 that can be configured to select the amplitude of the electrical stimulation between about 10 μA and about 15 mA. The amplitude selector 220 can also select whether the amplitude of the electrical stimulation is current driven or voltage driven. The electrode selector 225 can select to which of the electrodes 160 the electrical stimulation is delivered. The electrode selector 225 can also select which of the electrodes 160 are used as stimulating electrodes and which of the electrodes 160 are used as recording electrodes. Any of the above described selectors can be configured as software, scripts, or applications executed by the microprocessor 205.

The IPG 110 also includes a digital to analog (D/A) convertor 230. The D/A converter 230 is configured to output the electrical stimulation signals to an output stage 235. The output stage 235 can amplify the analog signal, change the impedance of the signal, filter, or otherwise change the characteristics of the signal. The output stage 235 can then direct the analog signal to the electrodes 160 as a stimulation signal. The IPG 110 can be configured to capture and record electrical signals from the target tissue. The IPG 110 includes a pre-amplifier 245. The pre-amplifier 245 amplifies the signals captured by the electrodes 160 and provided to the IPG 110. The signals are captured as analog signals that are converted to a digital signal by an analog to digital (A/D) convertor 240. The digitized signal can then be stored in the memory 250. The microprocessor 205 can retrieve signals stored in the memory 250 and transmit the signals to an external computer or display for viewing by a physician or healthcare professional. The memory 250 can also store programs, scripts, applications, and procedures, executed by the microprocessor 205.

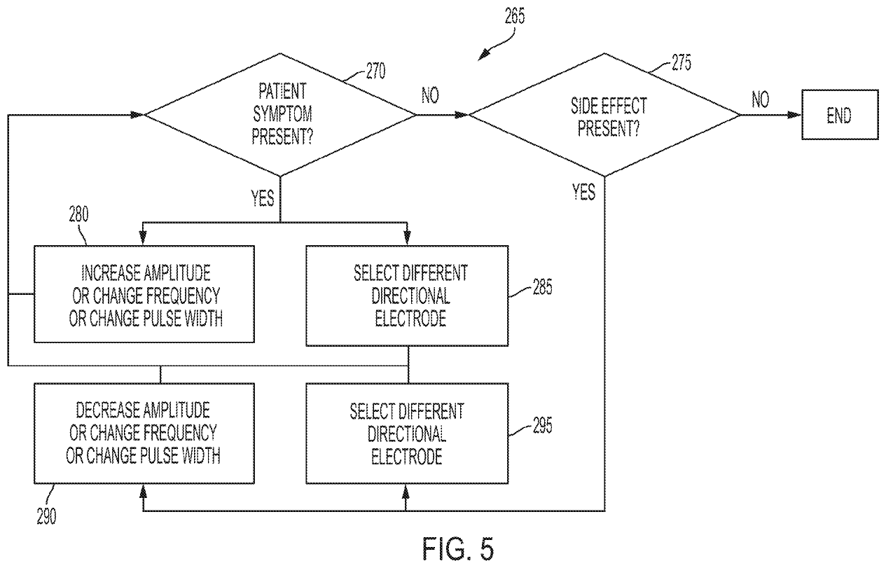

FIG. 5 illustrates a flow chart of a method 265 for tuning the electrical stimulation delivered to a patient. The method 265 includes determining if symptoms of the disorder are present (step 270). If symptoms are present, the method 265 can include selecting a different one of the electrodes used to stimulate the patient (step 285). The method 265 can also include increasing one or more of the characteristics of the electrical stimulation (step 280). After a predetermined amount of time, it can be re-determined whether the patient is experiencing symptoms (step 270).

As set forth above, the method 265 begins with the determination of whether a patient is experiencing symptoms of the disorder (step 270). Either prior to performing step 270 or after performing step 270, one or more electrode leads described herein can be implanted into or near a target location. The leads can be implanted by driving the leads towards the target location. The location of the lead can be confirmed using stereotaxic procedures, imaging procedures, and/or by making recordings with the lead to determine if characteristic signals from the target location are recorded. A second lead can be implanted at a second target location that can be on the contralateral side of the brain from the first target location.

One aspect of the present disclosure provides a newly identified ideal neural area or unique landing zone in the VMT/VTA (e.g., slMFB) that when stimulated with deep brain stimulation significantly reduced OCD symptoms when compared to stimulation in other areas of the brain. In addition to the suppression of OCD symptoms, stimulation of this ideal neural area or unique landing zone in the VMT/VTA (e.g., slMFB) also resulted in the quick suppression of depressive symptoms. The identification of this ideal neural area (e.g., unique landing zone) for implanting leads and electrodes for DBS stimulation within the VMT/VTA (e.g., slMFB) can support the idea of a shared disease-network and indicated that deep-brain stimulation of the ideal neural area or unique landing zone may result in the normalization of the reward network induced by the modulation of the slMFB-activity.

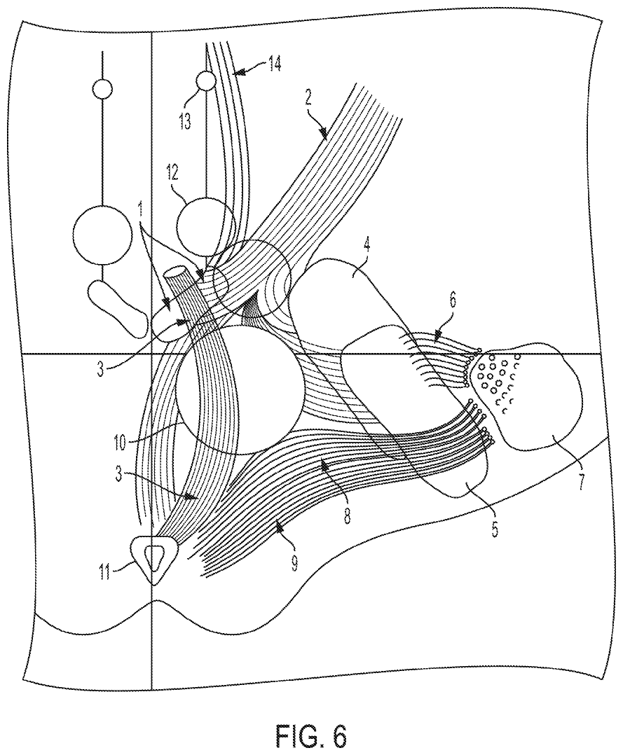

In the method disclosed herein, the ideal neural area or unique landing zone (e.g., the target location) can include the superolateral medial forebrain bundle (slMFB) 2 of the subject. As illustrated in FIG. 6, the slMFB 2 stimulated area is located in a crowded area of the brain (indicated by sphere between the mammillary-bodies 12, the red nucleus 10, and the anterior most aspect of the subthalamic nucleus 5). The electrical field can be delivered to the mesolimbic and the mesocortical pathway of the MFB of the subject. As shown, the target area is very close to the occulomotor nerve 3 (indicated by white arrows) that traverses the ventral tegmental area (“VTA”) 1 laterally. The electrical field can be adjusted to avoid the subject's hypothalamus, subthalamic nucleus 5, substantia nigra 4, occulomotor nerve 3, hyperdirect pathway 6, corticospinal tract 7, dentate-rubro-thalamic tract 8, medial lemniscus 9, red nucleus 10, periaqueductal grey 11, mammillary body 12, formix 13, inferomedial branch of the medial forebrain bundle (MFB) 14, or a combination of two or more thereof. The electrical field is adjusted to avoid stimulation of the occulomotor nerve 3. Avoiding these areas results in limited or reduced side effects. The electrical field may be delivered to the mesolimbic or the mesocortical pathway of the medial forebrain bundle of the subject. The method exhibits less disabling side effects compared to the same method conducted with an omnidirectional electrical field at the same pulse frequency, same pulse width, and/or same pulse amplitude. As used herein, “disabling side effects” or “side effects” include tonic muscular contraction, dysarthria, conjugate eye deviation, paresthesia, gait imbalance, behavioral impairments, limbic side effects, or a combination of two or more thereof.

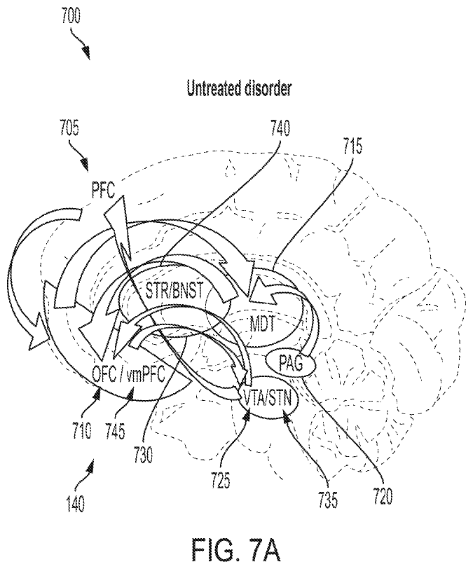

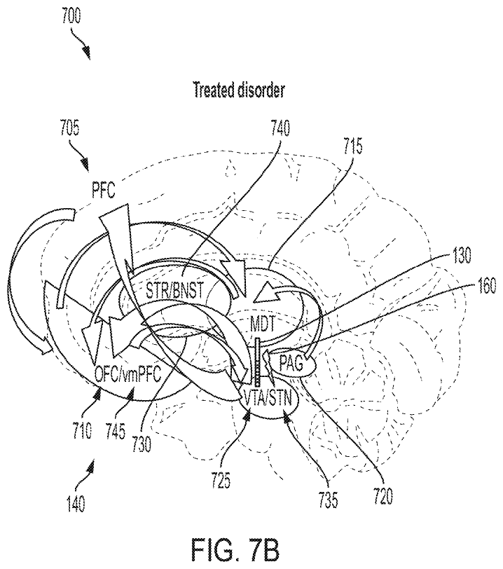

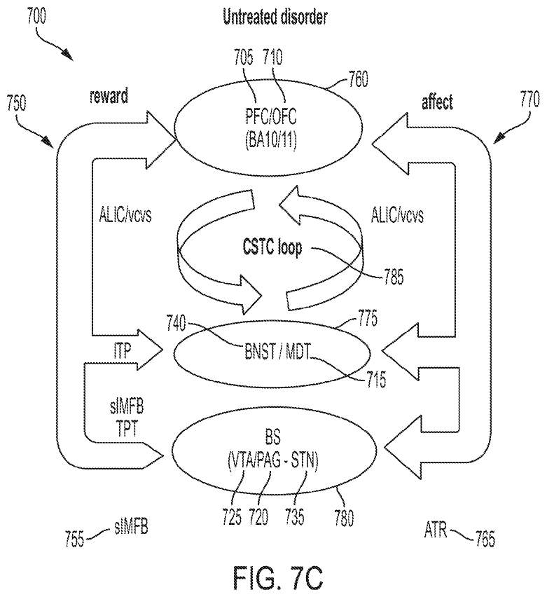

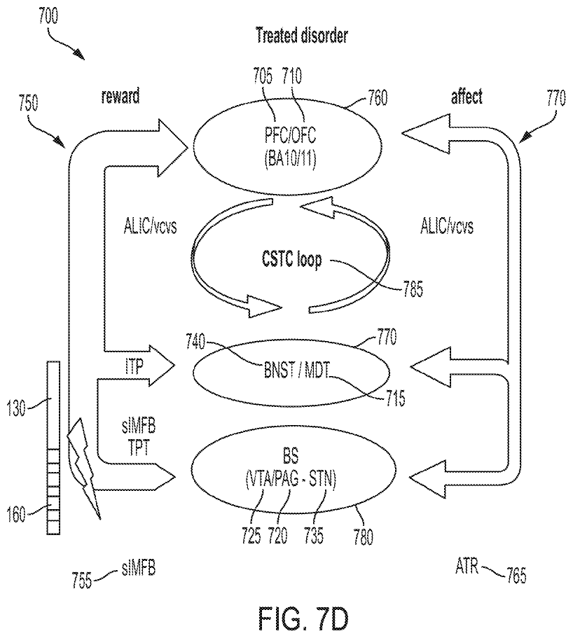

Not wishing to be bound by theory, as illustrated in FIGS. 7A-7D, treatment of psychiatric disorders (e.g., OCD) by electrical stimulation of the slMFB 755, VTA 725, or VMT modulates the hyperactive cortico-striato-thalamo-cortical circuits 785, which in turn results in reduced psychiatric symptoms. The circuit 700 comprises multiple components that are interconnected and may be involved in the pathogenesis of OCD. For example, the relevant neural components of the circuit 700 in a brain 140 of an OCD patient include: prefrontal cortex (PFC) 705; orbitofrontal cortex (OFC) 710; ventromedial prefrontal cortext (vmPFC) 745; striatum (STR) 730; be nucleus of stria terminalis (BNST) 740; mediodorsal thalamus (MDT) 715; periaquaeductal grey (PAG) 720; ventral tegmental area (VTA) 725; subthalamic nucleus (STN) 735; superolateral medial forebrain bundle (slMFB) 755. Many of these neural region may be tightly associated as shown in FIGS. 7A-B. For example, STR 730 may be tightly associated with BNST 740; OCF 710 may be may be tightly associated with vmPFC 745; and VTA 725 may be tightly associated with STN 735.

Thus, two major networks coverage onto the prefrontal cortex (“PFC”)—the reward network 750 (slMFB, VMT, VTA (1060)) and the affect network (ATR) 770. Together these networks form the cortico-striato-thalamo-cortical (“CSTC”) loop 785. The neural circuit 700 is misregulated in an OCD patient. In the brain 140 of an OCD patient, the cortico-striato-thalamo-cortical circuits 785 may be hyperactive creating an imbalance in the integration of neural interpretation of the reward circuit 750 with that of the affect circuit 770 as shown in FIG. 7C. This imbalance may in turn result in reduced psychiatric symptoms. The reward circuit 750 and the affect circuit 770 converge on the PFC/OFC 760 circuit, the CSTC loop 758, the BNST 740/MDT 715 circuit, and the BS 780. The BS 780 circuit comprises the VTA 725, the PAG 720, and the STN 735 regions. The reward circuit 750 may be mediated in part by the slMFB 755 and the affect circuit may be mediated by the ATR 765.

In psychiatric disorders, the affect network (ATR) 770 may be overactive and hyperconnected to e.g., the CSTC loop 758, and the BNST 740/MDT 715 circuit. DBS treatment of OCD as described herein can require placement of one or more leads 130 comprising one or more electrodes 160 in the VTA 725/SNT 735 region as shown in FIG. 7B. DBS stimulation may strengthen the reward circuit 750, while concomitantly suppressing the affective circuit 770, thereby treating and/or ameliorating OCD symptoms by, for example suppressing obsession 1305.

The reward network (slMFB) 755 acts as an opposing circuit. For example, the electrical stimulation of the slMFB 755, VMT, or VTA 725 (collectively circuit 1060) may upregulate the reward network 750 (slMFB, VMT, or VTA) and/or may downregulate the affect network (ATR) 770, which may have a tristolytic effect. See Coenen et al. NeuroImage Clinical 25:102165 (2020) (herein incorporated by reference).

The present disclosure provides a method for treating one or more psychiatric disorders. Examples of psychiatric disorders contemplated by the present disclosure include, but are not limited to, Attention-deficit and hyperactivity disorder (ADHD), Attention-deficit disorder (ADD), obsessive-compulsive disorder (OCD), anxiety, depression, a learning deficiency, an attention related deficiency or dysfunction, amnesia, a memory dysfunction, traumatic brain injury, stroke, dementia, neurodegenerative disorder, or Therapy refractory depression (TRD). The psychiatric disorder can be TRD or OCD. Alternatively, the psychiatric disorder can be OCD.

TRD and OCD may be characterized by deficiencies in showing flexible behavior. See e.g., Coenen et al., Brain Sci. 12:438 (2022); and Coenen et al., Brain Struct. and Funct. 227 (1): 23-47 (2022). Indeed, OCD may be associated with a deficiency in the automated classification of emotion related information (affect valence). As such, TRD and OCD may be considered as network dysfunctions of the MFB.

The MFB is an anato-physiologically region of the brain that regulates emotion (reward system) and controls of emotion associated bodily reactions, motor display of positive affect and motor learning. In particular, the MFB is associated with the seeking system, which promotes euphoric drive, reward anticipation, and reward. This seeking system is mediated in part by the activation of the mesocorticolimbic dopaminergic system.

Inventors of the present disclosure unexpectedly found that, in addition to regulating the reward system, the MFB may also serve an overarching role in the maintenance system. The maintenance system governs the valence of basal emotional states (e.g., aversive vs. appetitive). The maintenance system may also drive emotional arousal, basic and higher affect valence, bodily reactions, motor programs, vigor, and flexible behavior. This regulatory role basis of the high efficiency of deep brain stimulation (DBS) of the superolateral medial forebrain bundle (slMFB) as described herein. The overarching role of the slMFB in the maintenance system can be the basis for the antidepressant and anti-OCD efficacy of deep brain stimulation of the unique landing zone 1060 in the VMT/VTA described herein.

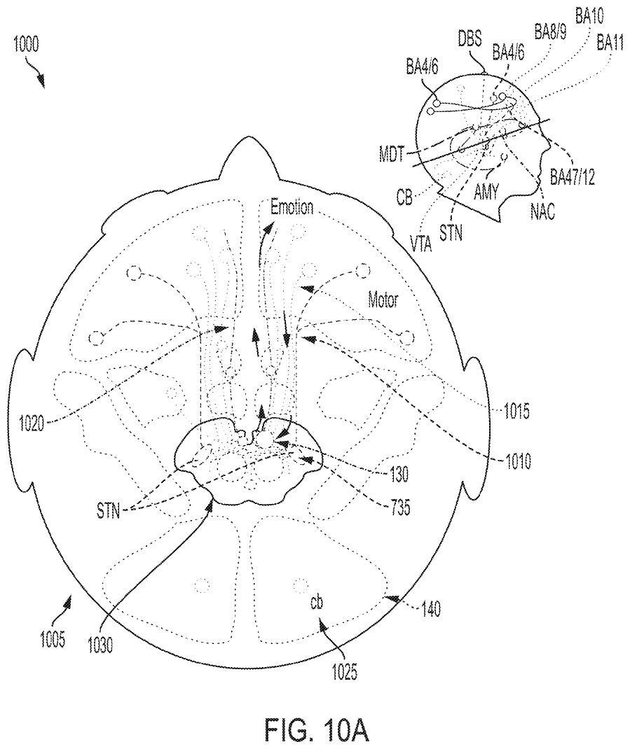

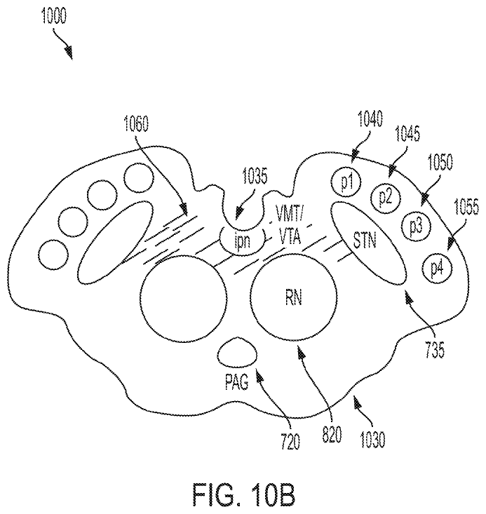

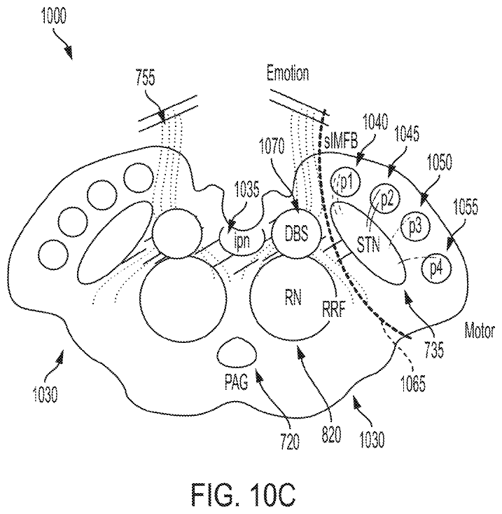

The maintenance system plays an essential role in the generation of flexible behavior. Dysfunction of the maintenance system may be associated with pathological alterations that underlie many psychiatric disorders (e.g., OCD and TRD). The maintenance system is illustrated in FIGS. 10A-C. FIG. 10A shows a schematic 1000 of a brain 140 with the Maintenance circuitry (1020, and 1015). Internal fibers 1020 represent the inferomedial MFB (imMFB) and the principal Dopaminergic (DA) projections to cortical and subcortical structures that may control emotion. Inner fibers 1015 represent cortical and cerebellar 1025 feedback projections to the ventral tegmental area (VTA) 725, which are Glutamatergic. External fibers 1065 illustrate part of the motor control system. The motor control system 1065 may have projections from the lateral orbitofrontal cortex (IOFC) 710 and dorsal prefrontal cortex (dlPFC) 705 to the subthalamic nucleus (STN) 735, which are also Glutamatergic (hyperdirect pathway).

The VTA 725 may be an evolutionarily ancient structure that may be closely connected to Papez' episodic memory circuitry via the mammillary bodies (MBB). The behavioral arousal associated with the VTA 725 may be accompanied by immediate bodily arousal that prepares the organism for a motor response. For these reasons, the maintenance system 1000 may inherently foreshadow such a motor response. Once behavioral arousal and basic affect valence have occurred, higher affect valence and interpretation of interoceptive and exteroceptive signals takes place and basic emotions are triggered. These systems are realized via distinct and proprietary higher systems with anatomical architectural expression (and also as part of cortical networks). Accordingly, higher affect valence (1020, and 1015) leads to the initiation of a motor response 1065. In a top-down mechanism, the dlPFC 705 can at any time control and moderate the execution of emotionally driven motor programs (via e.g., STN 735 activation), resulting in flexible behavior. As such, emotionally driven and speedily initiated behavior remains under dynamic frontal lobe (e.g., PFC 705 and/or OFC 710) top-down control.

As shown in FIG. 13, model 1300, in an OCD brain, intrusive and ego-dystonic thoughts enter the system as an interoceptive signal (e.g., obsession 1305). These thoughts are aversive and trigger meaningless motor programs (e.g., compulsions 1310). Such a high level of emotionally induced motor activity may allow only reduced flexible behavior with respect to obsessions. Alternatively, in a TRD brain, this maintenance system may be degraded. See e.g., Coenen et al., Brain Sci. 12:438 (2022); and Coenen et al., Brain Struct. and Funct. 227 (1): 23-47 (2022).

Thus, the present disclosure shows for the first time that a distinct neural circuit (e.g., VTA/VMT/slMFB 1060) modulates the emotional component (e.g., obsessions 1305) of OCD and a different neural circuit (e.g., STN 735/amSTN) modulates the motor component (e.g., compulsions 1310) of OCD. These distinct pathways are illustrated in FIG. 13, which shows a model whereby the PRF, VMT, VTA, and/or slMFB (collectively 1060) specifically may modulate the obsession component (emotional consequences) of OCD without affecting its compulsion component (motor consequences of the behavior); whereas the SNT 735/amSNT may control the motor consequences of these emotions.

One aspect of the present disclosure provides a method of treating one or more psychiatric disorders in a subject in need thereof comprising the DBS stimulation of the circuit 1060 (SIMFB, VTA, VMT, or pre-rurbral fields (PRF)) to specifically target and reduce the obsession component (emotional consequences) of OCD without affecting its compulsion component (motor consequences of the behavior). For example, DBS cannot stimulate the STN 735/amSTN.

FIG. 10B shows schematic 1000 illustrating an enlarged midbrain 1030 topographic overview from FIG. 10A. The hatched region 1060 (VTA/VMT) represents the principal origin of the dopaminergic (DA) fibers (A10 and medial A9 cell groups), which may be stimulated for the method of treatment disclosed herein. The placement of electrodes 160 or leads 130 specifically in this region 1070 can be important for treating OCD without underlying motor side effects as described herein. In a normal brain, these dopaminergic (DA) fibers (A10 and medial A9 cell groups) fibers modulate basal behavioral arousal, which leads to a basic affect valence (e.g., appetitive < > aversive). The DA groups A10, 9, and 8 contribute to reward-related behavioral arousal and can be activated through stimulation of descending glutamatergic fibers that enter the VTA 725. Placement of the DBS electrodes in this region 1070 can silence arousal system in patients with OCD or TRD. The reduced arousal and/or conscious focus may dampen obsession and agitation, thereby treating OCD (e.g., anti-OCD effect). Accordingly, in the method disclosed herein, chronic stimulation of the VTA 725 DA neuron population can have an anti-OCD effect because DBS feeds a distinct frequency spectrum into the maintenance system. For example, DBS of 1060, which comprises the VTA, VMT, and/or slMFB, can alter high frequency bursting state in OCD brain 140, which can be perceived as an anxiety signal by the prefrontal cortex (PFC) 705 resulting in OCD behaviors.

FIG. 10C shows a schematic 1000 illustrating the midbrain 1030 topographic overview as shown in FIG. 10B illustrating the slMFB 755 fibers with descending fibers diverging in front of the red nucleus (RN) 820 to reach the VTA 725 and raphe as well as the lateral tegmentum (RRF, retro-rubral field, DA group A8). The slMFB 755 neurons are not doparminergic, however, the slMFB 755 may modulate midline dopaminergic pathways (imMFB) through slMFB's corticofugal glutamatergic (Glu) projections to the midbrain tegmentum and VTA 725. The slMFB 755 can suffice as part of a maintenance circuit system that feed into the ventral tegmental area (VTA) 725 via a glutamatergic signal. The glutamatergic signaling triggered by slMFB DBS 1070 can play an important role in the observed antidepressant and anti-OCD effects.

The superolateral medial forebrain bundle (slMFB) 755 is a white matter structure connecting established targets for deep brain stimulation (DBS) in obsessive-compulsive disorder (OCD). slMFB 755 is a subsection of the medial forebrain bundle (MFB). DBS of SIMFB 1070 may be an accepted treatment strategy for TRD and/or OCD. However, DBS shows severe side effects, such as dyskinesia, diplopia, and/or hypomania if the DBS electrodes are not carefully and properly placed in the target area 1060. As such, correct placement of DBS electrodes on the slMFB 755 fibers can have anti-OCD effects without related side effects (e.g., motor side effects).

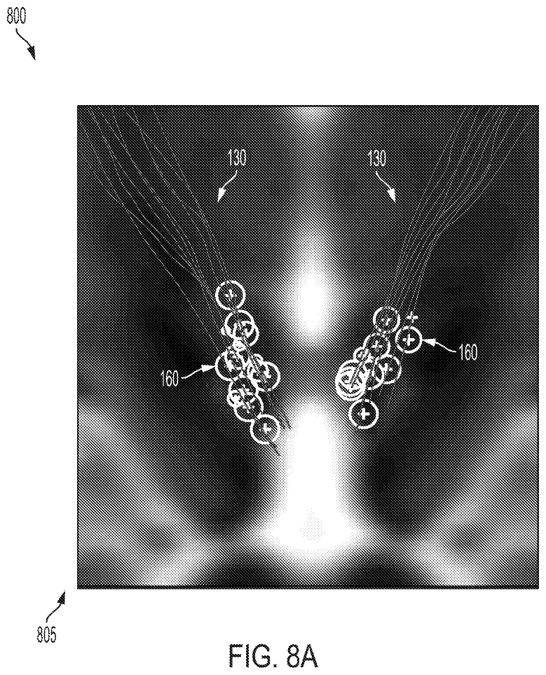

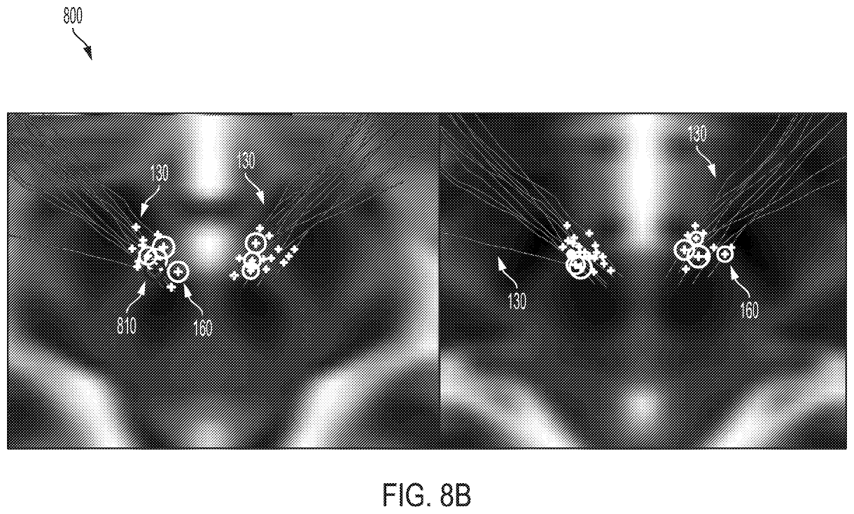

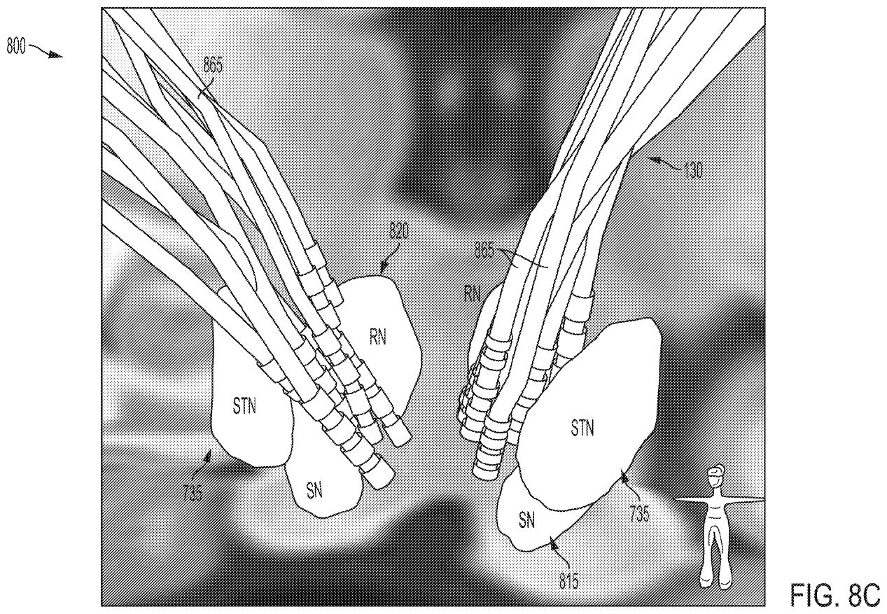

FIGS. 8A-C show coronal and axial views of template contacts 805 illustrating the careful placement of DBS electrodes 160 to specifically stimulate the slMFB 755 without impacting the motor system 1075, STN 735, or the anteromedial subthalamic nucleus (amSTN). In particular, the coronal and/or axial views of template contacts 805 show the positions of implanted electrode 160 within the unique landing zone 1060 identified herein (e.g., the mesencephalic ventral tegmentum (VMT), VTA 725 or slMFB 755) for treating OCD in patients. FIG. 8A shows a coronal view and FIG. 8B shows an axial view of magnetic resonance imaging (MRI) of electrode 160 contacts implanted within the target region 1060 of a patient. FIG. 8C shows a three-dimensional reconstruction of the positions of implanted active leads 130 and non-responder leads 865 in relation to the STN 735, RN 820 and SN 815.

As shown in FIGS. 8A-C, all leads 130 or electrodes 160 can be located in the ventral tegmentum 725 and anterior to the red nucleus (red) 820 and medial of the subthalamic nucleus 815. DBS of the 1060 region, which includes slMFB, VMT, or VTA (e.g., unique landing zone), can significantly reduce OCD symptoms when compared to DBS of non-SIMFB, VMT, or VTA region. DBS of the slMFB 755 can also significantly reduce depressive symptoms in OCD patients.

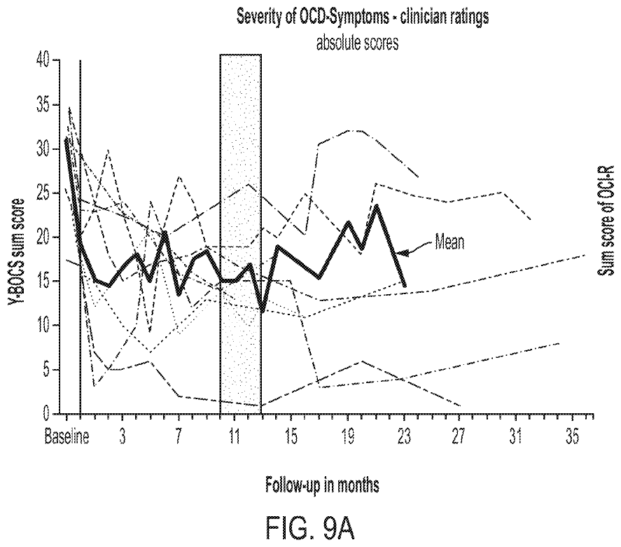

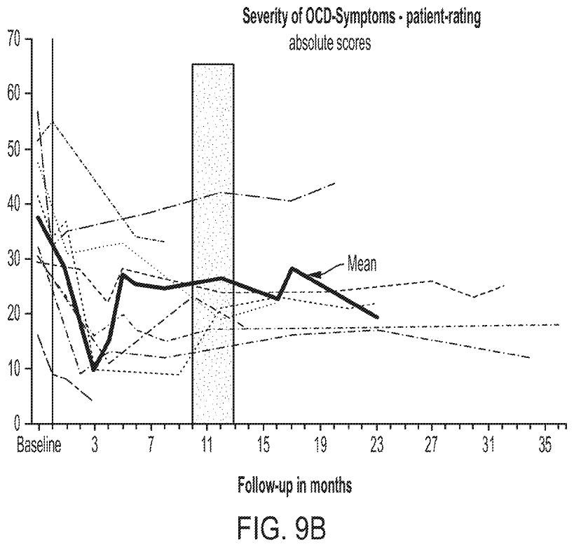

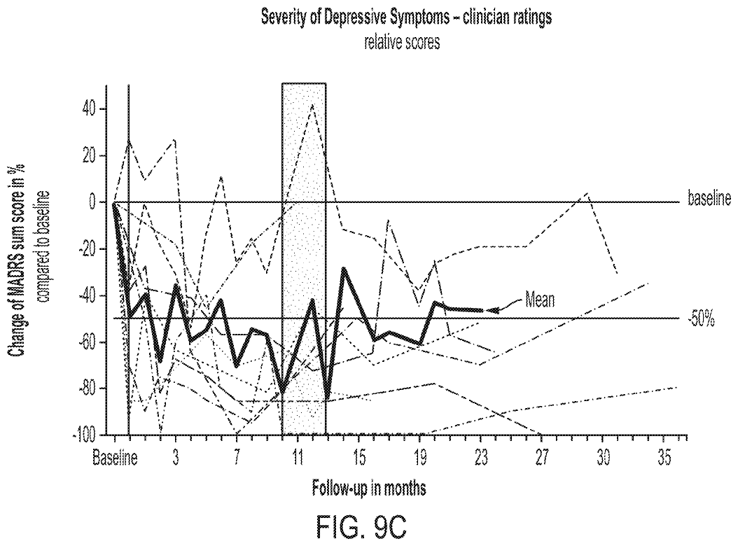

For Example, FIGS. 9A-C show relative changes in the OCD symptom severity over time for each patient after the placement of DBS electrodes on the slMFB when compared to baseline using the Yale-Brown obsessive compulsive scale (Y-BOCS sum scores). The large anti-OCD effect, the long-term efficacy data, and the safety-profile of slMFB DBS observed by placing the DBS electrodes on the ideal neural area (e.g., unique landing zone) identified herein were unexpected. This is mainly because the STN was believed to play an important role in DBS-mediated anti-OCD effects. Accordingly, the present disclosure shows for the first time that slMFB DBS may normalize the reward network that is modulated by the maintenance neural circuit and the STN is not required.

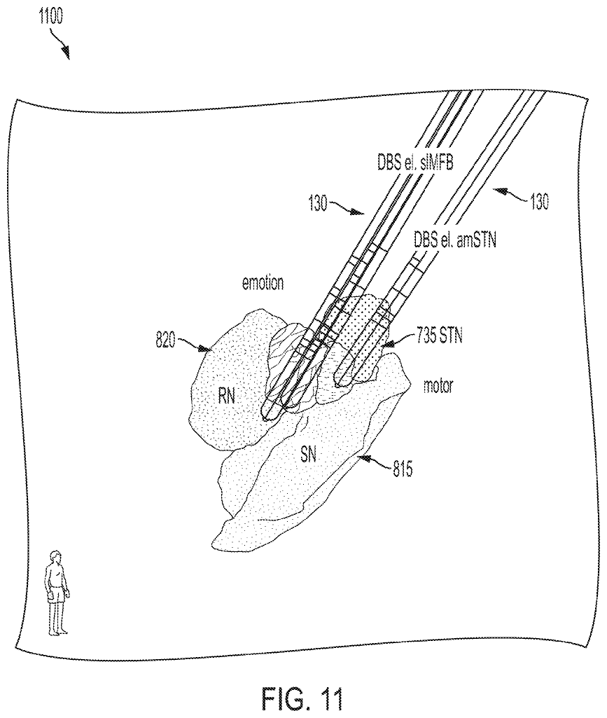

In particular, the method of treating a psychiatric disorder using slMFB, VMT, or VTA DBS described herein does not result in motor side effects (e.g., stimulation-induced dyskinesia). A plethora of DBS targets have been studied for OCD. As described herein, the great majority of these targets induce stimulation-mediated motor side effects (e.g., fine motor disturbance). The present disclosure shows for the first time that the placement of DBS electrodes on slMFB and away from the amSTN medial can prevent motor disturbance (e.g. dyskinesias) in OCD patient while maintaining potent anti-OCD efficacy (Example 3). This effect is particularly significant when the DBS electrodes and/or stimulation were steered toward the direction of the mesencephalic ventral tegmentum (VMT) which contains the ventral tegmental area (VTA), and the dopaminergic system disclosed herein. FIGS. 11 and 12A-12C show that the correct placement of DBS electrodes on the slMFB 755 and a bilateral stimulation preserved potent anti-OCD efficacy. The region just medial and outside the STN 735 produced strong anti-depressant and anti-OCD effects upon stimulation with the DBS technology. However, general stimulation of this area may also induce severe side effects such as hypomania or dyskinesia. This is because the proximity of the STN and slMFB makes it difficult to distinguish the role of the STN and slMFB in the generation of DBS-induced anti-OCD effect and/or motor activity. For example, FIG. 11 shows a 3D simulation of the positions of two DBS leads 130 (e.g. electrodes) with respect to the STN 735 in the targeted area. In particular, the tip of a lead 130 that stimulates the STN 725 (DBS el. amSTN) is very closed to the lead 130 that stimulates the slMFB 755 (DBS el. slMFB). Using one or more segmented electrodes disclosed herein can address this problem as described herein.

FIGS. 12A-12C show postoperative computed tomography three-dimensional (3-D) reconstruction images of a simulation of Deep brain stimulation (DBS) settings in a patient suffering from dyskinesia before (FIGS. 12A-B) and after electrode reprograming to fully avoid the STN 735 (e.g., amSTN) and focus on the landing zone 1060, which comprises VMT, VTA 725 and slMFB 755 (FIG. 12C). Following an initial electrode placements, the OCD symptoms in the patient were reduced, but the patient suffered from troublesome dyskinesia to her right leg. A simulation of the volume of activated tissue patterns revealed a co-stimulation of the patient's left anteromedial STN 735 or subthalamic nucleus 815, which may have potentially corresponded to the patient's troublesome dyskinesias. For example, FIG. 12A shows the placement of electrodes in the patient prior to any adjustment demonstrating that right-sided stimulation was fully medial and inferior to the STN 735. FIG. 12B shows the placement of electrodes in the left-side prior to any adjustment and shows the lead 130 (e.g. VAT) touches the STN 735 at position 1215 (dotted line). The contact at position 1215 may trigger dyskinesia. As such, the DBS electrodes (e.g., lead 130 or electrode 160) may co-stimulate the STN 735 or amSTN when not focused on the landing zone 1060, which includes slMFB 755, VMT or VTA 725.

If the position is not initially correct or motor side effects are observed, the left or right lead 130 (or electrode 160) may be reprogrammed to stimulate more distally along the electrode and to steer the stimulation away 1210 from the STN 735 or the anteromedial STN 735 and more towards 1060 (e.g., the VMT/VTA and the slMFB). Following reprograming, the patient may show an immediate remarkable and sustained motor improvement. The dyskinesias may be resolved within hours (based on e.g., UPDRS IV, subscore A, after reprogramming 0/8, no dyskinesias) and previous left- or right-sided fine motor disturbances may be gone. In addition, the patient's mood may be improved. FIG. 12C shows the placement of the DBS electrodes on the unique landing zone 1060 (e.g., ideal neural area or unique landing zone for implanting leads and electrodes for DBS stimulation within the VMT/VTA (e.g., slMFB)) after reprogramming. When placed on the ideal neural area or unique landing zone, cortical involvement of the left-sided stimulation may show minimally reduced engagement of the lateral inferior frontal gyrus with enhanced engagement of the slMFB. Thus, altering DBS settings (e.g., settings: 1 positive, 2 negative 25%, 4 negative 75%, 3.7 mA) to steer away from the STN 735 to focus on the ideal neural area 1060 identified herein can be sufficient to alleviate dyskinesias, while preserving strong anti-OCD efficacy. Focusing DSB electrodes on the slMFB (e.g., ideal neural area or unique landing zone) can be sufficient for anti-OCD efficacy.

Accordingly, the present disclosure demonstrates for the first time that the anti-OCD network targeted by DBS may be located outside and medial to the STN. The anti-OCD network targeted by DBS may be located in the VMT. Alternatively, the anti-OCD network targeted by DBS can be located in the VTA. These observations were unexpected because the prior art disclosed that the most efficient anti-OCD network targeted by DBS was located at the border of the white matter, which is medial to the nucleus (VMT or VTA) based on the efficient stimulation of the amSTN.

Accordingly, one aspect of the present disclosure provides a method for treating one or more psychiatric disorders selected from the group consisting of Attention-deficit and hyperactivity disorder (ADHD), Attention-deficit disorder (ADD), obsessive-compulsive disorder (OCD), anxiety, depression, a learning deficiency, an attention related deficiency or dysfunction, amnesia, a memory dysfunction, traumatic brain injury, stroke, dementia, neurodegenerative disorder, and Therapy refractory depression (TRD). The psychiatric disorder can be TRD or OCD. Preferably, the psychiatric disorder can be OCD.

The psychiatric disorder contemplated herein can be resistant to chemical treatment and/or psychotherapy treatment. For example, the psychiatric disorder can include depression, sleep disorder, anxiety disorder, anorexia nervosa, post-traumatic stress disorder, or a combination of any two or more thereof. Preferably, the depression is major depressive disorder.

The psychiatric disorder can also include sleep disorder, anxiety disorder, anorexia nervosa, post-traumatic stress disorder, or a combination of any two or more thereof. For instance, the psychiatric disorder can include anxiety disorder, anorexia nervosa, or a combination thereof. For example, the psychiatric disorder can include anxiety disorder. In that case, the anxiety disorder can include obsessive compulsive disorder (“OCD”), panic disorder, generalized anxiety disorder, phobia, separation anxiety disorder, or a combination of any two or more thereof. Preferably, the anxiety disorder can include OCD.

The present disclosure also provides a method of treating obsessive compulsive disorder in a subject in need thereof. The method includes implanting one or more leads into the subject, wherein each of the one or more leads comprises a plurality of substantially rounded electrodes; generating an electrical signal; generating an electrical field exhibiting a pulse amplitude of 0.05 mA to 5 mA; and delivering the electrical field to a slMFB, VMT, or VTA (e.g., ideal neural area or unique landing zone) of the subject via at least one of the plurality of electrodes. The electrical field can have a directionality of at least 50°, a focal radius of 0.5 mm to 3.5, and exhibits a volume of tissue activated of 1 mm³ to 50 mm³.

The present disclosure also provides a method for treating a blood pressure disorder. Alternatively, the present disclosure provides a method for treating a psychiatric disorder as disclosed herein and a blood pressure disorder. The blood pressure disorder may include hypertension.

Various implementations of the microelectrode device have been described herein. These embodiments are given by way of example and not to limit the scope of the present disclosure. The various features of the embodiments that have been described may be combined in various ways to produce numerous additional embodiments. Moreover, while various materials, dimensions, shapes, implantation locations, etc. have been described for use with disclosed embodiments, others besides those disclosed may be utilized without exceeding the scope of the disclosure.

Devices described herein as either acute or chronic can be used acutely or chronically. They may be implanted for such periods, such as during a surgery, and then removed. They may be implanted for extended periods, or indefinitely. Any devices described herein as being chronic may also be used acutely.

One or more or any part thereof of the techniques described herein can be implemented in computer hardware or software, or a combination of both. The methods can be implemented in computer programs using standard programming techniques following the method and figures described herein. Program code is applied to input data to perform the functions described herein and generate output information. The output information is applied to one or more output devices such as a display monitor. Each program may be implemented in a high level procedural or object oriented programming language to communicate with a computer system. However, the programs can be implemented in assembly or machine language, if desired. In any case, the language can be a compiled or interpreted language. Moreover, the program can run on dedicated integrated circuits preprogrammed for that purpose.

Each such computer program can be stored on a storage medium or device (e.g., ROM or magnetic diskette) readable by a general or special purpose programmable computer, for configuring and operating the computer when the storage media or device is read by the computer to perform the procedures described herein. The computer program can also reside in cache or main memory during program execution. The analysis, preprocessing, and other methods described herein can also be implemented as a computer-readable storage medium, configured with a computer program, where the storage medium so configured causes a computer to operate in a specific and predefined manner to perform the functions described herein. The computer readable media can tangible and substantially non-transitory in nature, e.g., such that the recorded information is recorded in a form other than solely as a propagating signal.

A program product can include a signal bearing medium. The signal bearing medium may include one or more instructions that, when executed by, for example, a processor, may provide the functionality described above. In some implementations, signal bearing medium may encompass a computer-readable medium, such as, but not limited to, a hard disk drive, a Compact Disc (CD), a Digital Video Disk (DVD), a digital tape, memory, etc. In some implementations, the signal bearing medium may encompass a recordable medium, such as, but not limited to, memory, read/write (R/W) CDs, R/W DVDs, etc. In some implementations, signal bearing medium may encompass a communications medium such as, but not limited to, a digital or an analog communication medium (e.g., a fiber optic cable, a waveguide, a wired communications link, a wireless communication link, etc.). Thus, for example, the program product may be conveyed by an RF signal bearing medium, where the signal bearing medium is conveyed by a wireless communications medium (e.g., a wireless communications medium conforming with the IEEE 802.11 standard). Any of the signals and signal processing techniques may be digital or analog in nature, or combinations thereof.

The various concepts introduced above and discussed in greater detail below may be implemented in any of numerous ways, as the described concepts are not limited to any particular manner of implementation. Examples of specific implementations and applications are provided primarily for illustrative purposes.

The following terms are used throughout as defined below.

As used herein, the term “and/or” means any one of the items, any combination of the items, or all of the items with which this term is associated.

As used herein, singular articles such as “a”, “an”, and “one” are intended to refer to singular or plural. As such, this statement is intended to serve as antecedent basis for use of such exclusive terminology as “solely,” “only,” and the like in connection with the recitation of claim elements, or use of a “negative” limitation.

As used herein, the term “About” or “substantially” include the identified numbers and can refer to a variation of up to ±10% of the value specified. For example, “about 50” can carry a variation from 45 to 55 percent. “About” can also refer to a variation of ±1%, ±2%, or ±5%. Unless indicated otherwise herein, the term “About” is intended to include values, e.g., weight percentages, proximate to the recited range that are equivalent in terms of the functionality of the individual ingredient, the composition, or the embodiment. In addition, unless indicated otherwise herein, a recited range (e.g., weight percentages) includes each specific value, integer, decimal, or identity within the range.

As used herein, the term “Maintenance system” refers to neuronal circuitry that governs the valence of basal emotional states (e.g., aversive vs. appetitive) and serves to drive emotional arousal, basic and higher affect valence, bodily reactions, motor programs, vigor and flexible behavior, accordingly.

As used herein, the term “Subject” or “Patient” refers to a mammal, such as a cat, dog, rodent or primate. Typically the subject is a human, and, preferably, a human having or suspected of having a psychiatric disorder and/or blood pressure disorder. The term “Subject” and “Patient” can be used interchangeably.

As used herein, the term “Substantially directional” means not omnidirectional.

The term “Substantially focused” as used herein means the electrical field exhibits a volume of tissue activated of ≤55 mm³.

The term “Substantially rounded” as used herein means does not have sharp corners (e.g., corners where a vertex is met by largely perpendicular sides, or corners with a radius of curvature less than 100 micrometers).

As used herein, the term “Treating” or “Treatment” within the context of the present technology, means an alleviation, in whole or in part, of symptoms associated with a disorder or disease, or slowing, or halting of further progression or worsening of those symptoms.

As used herein the terms “Volume of activated tissue” “Stimulation field map” (SFM), “Volume of activation”, or “volume of tissue activated (VTA)” refers to the estimated stimulation region of tissue that will be stimulated for a particular set of stimulation parameters. Any suitable method for determining the VOA/SFM/VTA can be used including those described in, for example, U.S. Pat. Nos. 8,326,433; 8,675,945; 8,831,731; 8,849,632; and 8,958,615; U.S. Patent Application Publications Nos. 2009/0287272, all of which are incorporated herein by reference in their entireties.

### EXAMPLES

The examples herein are provided to illustrate advantages of the present disclosure and to further assist a person of ordinary skill in the art with using the present technology. The examples should in no way be construed as limiting the scope of the present technology, as defined by the appended claims. The examples can include or incorporate any of the variations, aspects or aspects of the present disclosure as described herein. The variations, aspects or aspects described herein may also further include or incorporate variations of any or all other variations, aspects or aspects of the present technology.

### Example 1: Materials and Methods

### 1. Sample Selection

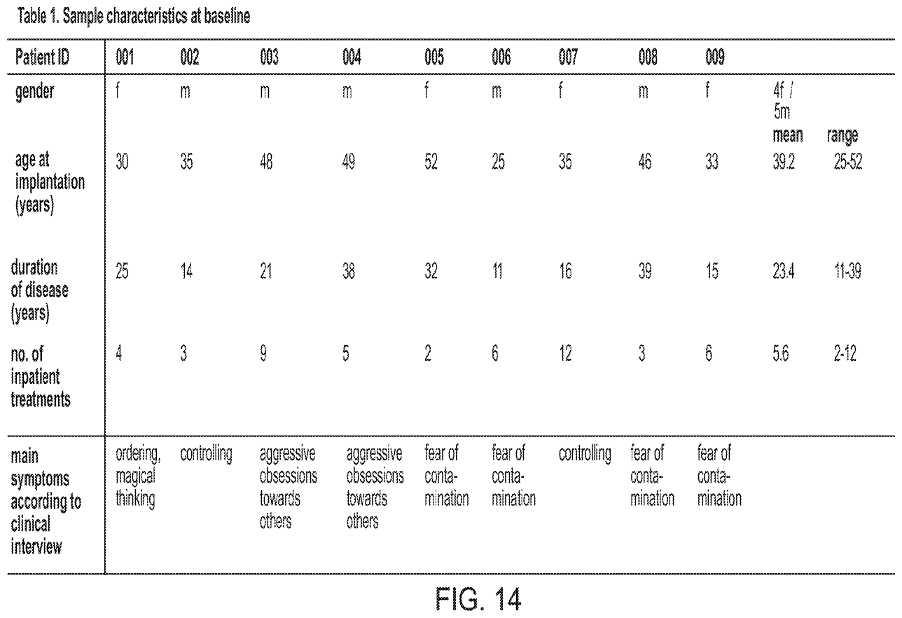

At least two comprehensive visits took place with each patient before admission into the experimental study. The comprehensive visits served to explore symptom severity as well as number, type and adequacy of prior treatments to assess the degree of treatment resistance. All patients were informed in detail about the risks and gave written informed consent prior to surgery and following treatment. All patients (10) gave informed consent to the scientific use of their clinical data and all but one gave consent to publication. FIG. 14 shows the clinical characteristics of the patient sample at baseline.

### 2. Measurements

Several clinician-rating and self-rating questionnaires were used to assess symptom severity. In all cases the corresponding validated German version were used. The Yale-Brown obsessive compulsive scale (Y-BOCS) was used to assess the severity of obsessions and compulsions. The Y-BOCS is a clinician-rating scale with a maximum sum score of 40 points (higher scores indicate higher severity). The subjective symptom severity was assessed with the Obsessive-Compulsive Inventory-revised (OCI-R). The OCI-R is an 18-item questionnaire with a maximum sum score of 72. Severity of depressive symptoms was assessed using the Montgomery-Åsberg Depression Rating Scale (MADRS). MADRS is a clinician-rating scale containing 10 items with a maximum sum score of 60. To assess severity of illness on a more general level as well as global level of functioning the Global Assessment of Functioning (GAF) (DSM-IV, axis V) was used. The GAF defines psycho-social and professional functioning from 0 (very severe psychopathology) to 100 (full mental health).

Data were assessed according to clinical routine. Patients were assessed about every third month, especially during the first year of stimulation. Visit frequency was then reduced according to clinical necessity. The stimulation devices were checked at every visit and stimulation settings adjusted if necessary. Patients did not keep concomitant medication and/or psychotherapy stable at all times.

### 3. Neurosurgery

A neurosurgical procedure including imaging, fiber tracking, surgery and postoperative care were conducted. All patients in the study received segmented electrodes (Vercise Cartesia, Boston Scientific). Eight patients received a rechargeable pulse generator (Vercise Gevia, Boston Scientific) and one patient received a non-rechargeable pulse generator (Vercise PC, Boston Scientific). The implantable stimulator was placed in the clavicle area in all cases. Implantations took place between December 2017 and January 2020.

### 4. Data Analysis

Clinical data (Y-BOCS, MADRS, GAF, OCI-R) were analyzed in a descriptive manner. Baseline scores were calculated as mean of all pre-implantation data available per patient (minimally 1 and maximally 4 pre-implantation scores). Baseline was defined as time from the first clinical visit until surgery. If multiple clinical ratings were available for one month, they were averaged. One year data contains the individual data point closest to 12 months after stimulation onset (range 10 to 13 months).

### Example 2: Stimulation of slMFB, VMT, or VTA in Patients Exhibiting OCD

Stimulation was initiated between 2 and 20 days after surgery (M=7.4 days). Stimulation settings were chosen based on patients' individual therapeutic window. Depending on this, either a unipolar or bipolar stimulation mode was preferred. Generally, initial programming settings were set to a bipolar stimulation mode with mean currents of 2 mA, a pulse width of 60 μsec and a frequency of 130 Hz. In total, currents ranged from a minimum of 1.4 mA to a maximum of 5.5 mA and pulse width ranged from 30 μsec to 150 μsec. Frequency was constantly set to 130 Hz. In case of therapeutic inefficiency, the current was titrated until the threshold of side effects was reached. In a second step a pulse width higher than 60 μsec was chosen to increase the total electric energy delivered. In one case, the pulse width was shortened down to 30 μsec to widen the therapeutic window. If necessary, contacts were switched or reevaluated for efficacy and side effect threshold. Generally, the contact with the largest therapeutic window was chosen for chronic stimulation. The initial programming of the stimulation device was performed shortly after implantation and further adjustments of stimulation settings were performed by two psychiatrists during follow-up. FIG. 8A (coronal plane image) and FIG. 8B (axial plane image) show the electrodes were implanted in the ventral tegmentum (anterior to the red nucleus (“RN”) and medial of the subthalamic nucleus (“STN”)). Active electrode contacts are indicated by spheres. FIG. 8C provides the three-dimensional positions of the implanted electrodes in MNI normative space and active contacts at three months after stimulation onset.

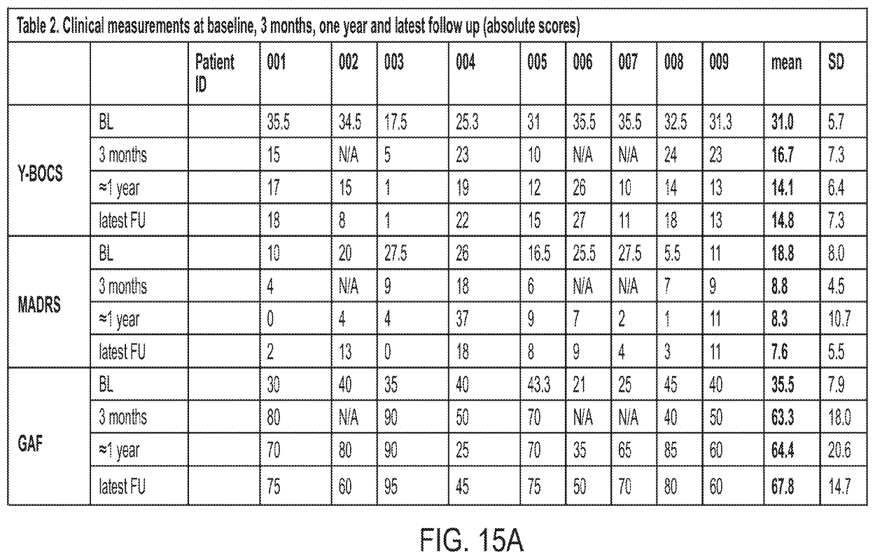

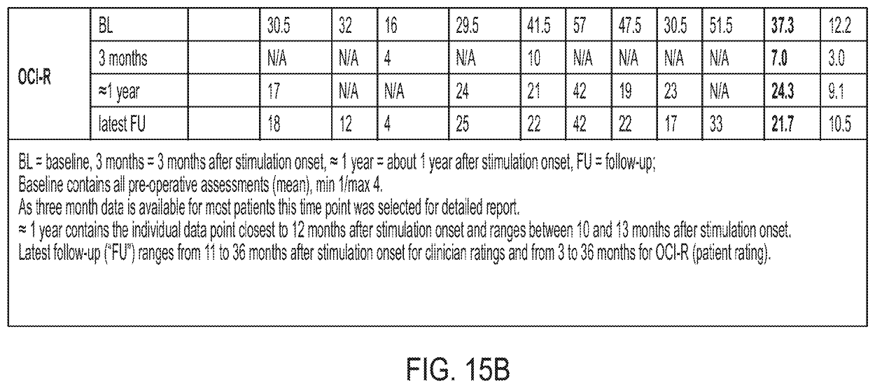

As shown in FIG. 9A, OCD symptoms decreased quickly with three out of nine patients responding at the first assessment after stimulation onset and >20% reduction of Y-BOCS sum score in three other patients. Mean duration until response criterion was reached for the first time was three months (ranging from stimulation onset to eight months). After one year of stimulation (range 10 to 13 months) seven patients were classified as responders and sustained response until last follow-up (ranging from 11 to 36 months of stimulation depending on date of surgery). Two patients responded only occasionally. At three-months (available for six out of nine patients), mean Y-BOCS sum score had decreased by 43.1% (SD=23.6, n=6) compared to baseline. The score had decreased by 55.9% (SD=20.0) after one year of stimulation and by 53.5% (SD=23.7) at last follow-up. In absolute numbers the data provides a mean reduction of 12.2 points (SD=6.7, n=6) at three months, 16.9 (SD=5.4) after one year, and 16.2 (SD=6.7) at last follow-up as indicated in FIGS. 15A-B. The BOCS obsessions scale and compulsion scale were also assessed separately and provided similar results (data not included).

Although patients 004 and 006 merely reached the response criterion, they still reported a decrease of symptoms (especially at the beginning of the treatment). Their symptoms fluctuated but stayed high at all times (FIG. 9B). Except patient 004, patients with comorbid moderate to severe depressive symptoms at baseline showed significant reductions in MADRS score. As shown in FIG. 9C, depressive symptoms fluctuated quite simultaneously with OCD symptoms. One patient (008) showed a mild increase in depressive symptoms immediately after stimulation onset without reaching a degree of clinical significance (MADRS<10).

The main limiting side effect was diplopia as a result of co-stimulation of oculomotor fibers running in close neighborhood to the lowest contact of the implanted electrode. These side effects were resolved immediately by adjustments of the stimulation parameters. All further adverse events are listed in Table 3.

TABLE 3
Medium term and persistent adverse events

| | No. of patients affected | |
|---|---|---|
| Adverse events | transiently | persistently |

Surgery related

| | | |
|---|---|---|
| epileptic seizure (later diagnosed as | 1 | |
| myoclonus-epilepsy) | | |
| fatigue | | 1 |
| loosening of a holding thread of the | 1 | |
| generator | | |
| (surgery required for fixation) | | |

Stimulation related

| | | |
|---|---|---|
| slightly blurred vision for about 1 month | 1 | |
| hypomania | 2 | |
| motoric difficulties regarding fine and | | 1 |
| gross motor skills | | |
| slurred speech | 1 | |
| inner restlessness | 1 | |
| frequent headaches | 2 | |
| stimulation cessation due to battery | 3 | |
| depletion | | |

Other

| | | |
|---|---|---|
| double vision immediately after | 1 | |
| stimulation reonset | | |
| reported increased trichotillomania | | 1 |

The results indicate the feasibility and safety of slMFB, VMT, or VTA electrical stimulation in severe, treatment-resistant OCD with strong and lasting therapeutic effect. FIGS. 15A-B demonstrate that a reduction of OCD symptoms went along with increased general functioning. At the latest assessment 5 patients reported at most mild, 2 moderate and 2 serious impairments compared to throughout serious impairments at baseline. Apart from the general improvement, symptom severity fluctuated over time in all patients (FIGS. 9A-9C). The clinical observation is that major life events do have impact on symptom severity even in responders. Patients 002 and 006 both experienced a major personal crisis and reported clearly increased OCD symptoms at the following visit (month 5 and month 16 respectively). However, symptom severity did not reach baseline levels and decreased again after some time. See e.g., Meyer et al., Brain Stimulation 15: 582e585 (2022)

These examples demonstrate that the overall reduction of symptoms is not the result of a general emotional numbness but that patients stay sensitive to events in their environment. Beyond OCD symptoms, eventually depressive symptoms decreased quickly after stimulation onset in seven patients.

### Example 3: The Anteromedial STN is not Necessary for Sustained DBS-Mediated Anti-OCD Efficacy

This example shows that anti-OCD efficacy in a patient was preserved and achieved with a bilateral stimulation of the slMFB which avoided the stimulation of the amSTN.

The anteromedial STN and slMFB, VMT, or VTA target regions are adjacent. To determine which of these two neural circuits may control OCD symptoms, the effect of slMFB DBS for OCD in the absence of a concurrent stimulation of the amSTN was determined.

The patient tested was a 52-year-old woman who reported obsessive and compulsive symptoms since her teenage years that exacerbated in early adulthood. Before DBS treatment, the patient suffered from an extreme fear of contamination, resulting in severe cleaning compulsions and strong avoidance behaviors. Consequently, the patient lived a very secluded life with a limited radius of action. Numerous pharmacological and psychotherapeutic, guideline-based treatment attempts over the last 20 years had shown none or insufficient success. The patient underwent uneventful bilateral implantation of directional DBS electrodes (Cartesia™, Boston Scientific, USA) connected to a subclavicular located pulse generator (Gevia RC™, Boston Scientific USA).

Bilateral implantation (Leksell G-Frame, Elekta, Sweden) was performed under microelectrode recording (MER) guidance to avoid the STN region (anterior, central trajectory). MER showed STN signal on the left side at target+5.5 mm. Right-sided MER showed no signal of any nucleus. Intraoperative testing below the STN level showed good anti-aversive effects. DBS electrodes (Cartesia, Boston Scientific, USA) were implanted bilaterally on the central trajectory. DBS electrode rotation was estimated for the left and right DBS electrodes with 40° to the left and 45° to the right, respectively (Guide XT™, Boston Scientific, USA and Elements, BrainLab, Munich). Stimulation was initiated bilaterally at 1.5 mA (60 μs, 130 Hz) in a bipolar setting (left: 1 pos, 2-4 neg 40%, 5-7 neg 60%; right: 2-4 pos 100%, 5-6 neg 90%, 7 neg 10%).

As shown in FIGS. 12A-C, postoperative computed tomography fused to preoperative MRI showed an optimal positioning of the DBS electrodes in the VMT. The patient experienced an immediate improvement ins obsessions and compulsions. For example, the mean baseline Y-BOCS sum score (three assessments pre-surgery) of 31 dropped to 16 two days after stimulation onset. OCD symptoms improved further resulting in a Y-BOCS sum score of 7 after 5 months of stimulation and 12 after 1 year.

However, fine motor disturbances affecting her right side were observed. Because of the superior improvement of OCD symptoms, the patient accepted the adverse effects. 32 months after DBS onset, the current amplitude was further increased (month 32:3.8 mA left, 3.6 mA right) to reduce mild depressive symptoms. In the following weeks, the patient reported troublesome changes of movement in her right leg with a concomitant loss of control and hyperactivity of the leg and foot. The patient showed intermittent activity-induced dyskinesias of her right foot and leg associated with a compensatory gait.

As shown in FIGS. 12A-B, a simulation of the volume of activated tissue patterns around the position of electrodes placement revealed a co-stimulation of her left anteromedial STN (subthalamic nucleus), potentially corresponding to the patient's troublesome dyskinesias. As shown in FIGS. 12B-C, the left DBS electrode was reprogrammed (month 35) after image-guided simulation of the volume of tissue activation patterns using its directional properties. The aim was to stimulate more distally along the electrode and to steer the stimulation away from the anteromedial STN, which may have been responsible for the induced dyskinesias and to steer the stimulation towards the VMT and the slMFB (FIG. 12C).

Following the reprogramming, the patient showed an immediate remarkable and sustained motor improvement and the dyskinesias resolved within hours (UPDRS IV, subscore A, after reprogramming 0/8, no dyskinesias). See e.g., Coenen et al., Acta Neurochirurgica 164:2303-2307 (2022). The patient's previous right-sided fine motor disturbance was also resolved. Her mood also improved, and she was well 4 months after change of settings (last observation, month 39; Y-BOCS sum score=10; MADRS sum=12).

Accordingly, the present example showed that anti-OCD efficacy in the patient was preserved and achieved with a bilateral stimulation, which fully avoided the STN. The present example further showed that the mesencephalic ventral tegmentum (VMT), slMFB, or pre-rurbral fields (PRF) constitutes an unique landing zone for the placement of DBS electrodes for the effective treatment of OCD and or TRD.

### EQUIVALENTS

While certain embodiments have been illustrated and described, a person with ordinary skill in the art, after reading the foregoing specification, can effect changes, substitutions of equivalents and other types of alterations to the compositions of the present disclosure as set forth herein. Each aspect and embodiment described above can also have included or incorporated therewith such variations or aspects as disclosed in regard to any or all of the other aspects and embodiments.

The present disclosure is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, or compositions, which can, of course, vary. It is also to be understood that the terminology used herein is for the purpose of describing particular aspects only, and is not intended to be limiting. Thus, it is intended that the specification be considered as exemplary only with the breadth, scope and spirit of the present disclosure indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

1. A method of treating a psychiatric disorder in a subject, comprising:
implanting one or more leads into the subject, each of the one or leads comprising a plurality of electrodes;
generating an electrical signal;
generating an electrical field; and
delivering the electrical field to a superolateral branch of a medial forebrain bundle (SIMFB), VMT, or VTA in the subject via at least one of the plurality of electrodes;
wherein the electrical field is substantially directional and substantially focused.

2. The method of claim 1, wherein each of the plurality of electrodes is segmented and does not have sharp corners.

3. (canceled)

4. The method of claim 1, wherein the one or more leads further comprises a MEMS film that comprises the plurality of electrodes.

5.-6. (canceled)

7. The method of claim 1, wherein the directionality of the electrical field is 20° to 50°.

8. (canceled)

9. The method of claim 1, wherein the focus of the electrical field has a focal radius of ≤4 mm.

10.-11. (canceled)

12. The method of claim 1, wherein the electrical field exhibits a volume of tissue activated of 1 mm³ to 50 mm³.

13. (canceled)

14. The method of claim 1, wherein the electrical signal exhibits a pulse amplitude of at least 0.05 mA.

15.-18. (canceled)

19. The method of claim 1, wherein the electrical field exhibits a pulse width range of about 1 μsec to about 500 μsec.

20.-21. (canceled)

22. The method of claim 1, wherein the electrical field exhibits a frequency of about 50 Hz to about 200 Hz.

23.-24. (canceled)

25. The method of claim 1, wherein the psychiatric disorder comprises any of depression, sleep disorder, anxiety disorder, anorexia nervosa, or post-traumatic stress disorder.

26.-28. (canceled)

29. The method of claim 1, wherein the anxiety disorder comprises obsessive compulsive disorder (OCD), panic disorder, generalized anxiety disorder, phobia, or separation anxiety disorder.

30.-32. (canceled)

33. The method of claim 1, further comprising treating a blood pressure disorder in the subject.

34. (canceled)

35. The method of claim 1, further comprising implanting an implantable stimulator into the subject, wherein the generating the electrical signal is generated by the implantable stimulator.

36. (canceled)

37. The method of claim 1, wherein the one or more leads have a MEMS film further comprises a plurality of periphery traces at least partially encircling each of the plurality of electrodes and at least two connection points coupling each of the plurality of periphery traces with a respective one of the plurality of electrodes.

38. (canceled)

39. The method of claim 1, wherein the delivering the electrical field is to a mesolimbic or mesocortical pathway of the medial forebrain bundle of the subject.

40. (canceled)

41. The method of claim 1, wherein the delivering the electrical field avoids the subject's hypothalamus, subthalamic nucleus, substantia nigra, occulomotor nerve, hyperdirect pathway, corticospinal tract, dentate-rubro-thalamic tract, medial lemniscus, red nucleus, periaqueductal grey, mammillary body, formix, inferomedial branch of the medial forebrain bundle, or a combination of two or more thereof.

42. The method of claim 1, wherein each of the one or more leads comprise 2 to 20 electrodes.

43.-44. (canceled)

45. A method of treating obsessive compulsive disorder in a subject in need thereof comprising:
implanting one or more leads into the subject, each of the one or more leads comprising a plurality of rounded electrodes;
generating an electrical signal;
generating an electrical field exhibiting a pulse amplitude of 0.05 mA to 5 mA; and
delivering the electrical field to a superolateral branch in a medial forebrain bundle of the subject via at least one of the plurality of electrodes;
wherein:
the electrical field has a directionality of at least 50°, a focal radius of 0.5 mm to 3.5, and exhibits a volume of tissue activated of 1 mm³ to 50 mm³.

46. A method of treating a blood pressure disorder in a subject in need thereof comprising:
implanting one or more leads into the subject, each of the one or more leads comprising a plurality of electrodes;
generating an electrical signal;
generating an electrical field; and
delivering the electrical field to a superolateral branch in a medial forebrain bundle in the subject via at least one of the plurality of electrodes;
wherein:
the electrical field is directional and focused.

47. The method of claim 46, wherein the blood pressure disorder comprises hypertension.
